(19) United States
(12) Patent Application Publication
(10) Pub. No.: US 2021/0061913 A1
(43) Pub. Date: Mar. 4, 2021

(54) PDL-1 INHIBITORS IN TREATMENT OF PULMONARY VASCULAR DISEASES

(71) Applicant: University of Florida Research Foundation, Incorporated, Gainesville, FL (US)

(72) Inventor: Andrew Justin Bryant, Gainesville, FL (US)

(21) Appl. No.: 17/006,452

(22) Filed: Aug. 28, 2020

Related U.S. Application Data
(60) Provisional application No. 62/894,376, filed on Aug. 30, 2019.

(51) Int. Cl. C07K 16/28; A61P 9/12

(52) U.S. Cl. CPC C07K 16/2827; A61P 9/12; C07K 16/2818

(57) ABSTRACT

The disclosure provides therapies that are useful in the prevention of pulmonary vascular remodeling and treatment of vascular conditions. In particular, the disclosure provides methods of treatment of pulmonary vascular diseases comprising administration of an inhibitor of programmed cell death protein 1 (PD-1) or the corresponding programmed death-ligand 1 (PD-L1). These methods comprise administering to a subject a PD-1 or PD-L1 inhibitor such as a monoclonal antibody. Further provided herein are diagnostic methods for assessing patient sensitivity to therapies. Methods of identifying a subject having a pulmonary hypertension that is sensitive to treatment with a PD-L1 inhibitor or a PD-1 inhibitor are provided. Subjects identified as having a pulmonary hypertension that is sensitive to anti-PD-L1 treatment may be subsequently administered a PD-L1 inhibition treatment.

### RELATED APPLICATIONS

This application claims the benefit under 35 U.S.C. § 119(e) of the filing date of U.S. Provisional Application Ser. No. 62/894,376, filed Aug. 30, 2019, the entire contents of which is incorporated herein by reference.

### GOVERNMENT SUPPORT

The invention was made with government support under Grant Nos. K08 HL144085 and R01 HL142776 awarded by the National Institute of Health. The government has certain rights in the invention.

### BACKGROUND

Patients with chronic lung disease complicated by pulmonary hypertension (PH; defined as World Health Organization [WHO] “Group 3 PH”) are faced with a difficult reality: no long-term treatment for this debilitating illness is available, outside of supplemental oxygen and lung transplant. Death often follows within a year. Therefore, novel targets for disease treatment are urgently required.

Myeloid-derived cells have previously been shown to be implicated in development of both pulmonary fibrosis and PH (Yeager et al., 2012). However, their mechanism of action is unknown. Proliferation of myeloid cells, specifically those with immunosuppressive capabilities, is known to contribute to aberrant vasculogenesis in several cancers, accounting for increased risk of uncontrolled cellular growth and metastasis (Al Sayed et al., 2019). Moreover, tumor-associated cytokine-signaling in this setting is known to provoke emergency myelopoiesis, an evolutionary response to states of chronic inflammation that is teleologically thought to protect the host against sustained immune activity or autoimmunity (Gabrilovich, 2017). Thus, a need exists for a safe, long-term treatment for pulmonary vascular diseases such as PH.

### SUMMARY OF THE INVENTION

Provided herein are novel methods of treatment of pulmonary vascular diseases and conditions. These methods comprise administering to a subject an inhibitor of programmed cell death protein 1 (PD-1) or the corresponding programmed death-ligand 1 (PD-L1)-targets having a newly understood association with pulmonary vascular symptoms, and in particular pulmonary hypertension (PH) and pulmonary arterial hypertension (PAH).

The present disclosure is based, at least in part, on the demonstration of an association between severity of pulmonary vascular conditions and circulating myeloid cell expression of PD-L1 in patients with pulmonary arterial hypertension. The present disclosure describes the effects of myeloid cell proliferation induced by myelopoeises on PH progression and the inhibitory effects conferred by administration of anti-PD-L1 therapies on myeloid cells that express PD-L1. In particular, the present disclosure shows that inhibition of PD-L1 binding in vivo unexpectedly confers a reversal of PAH-associated symptoms. The disclosure provides therapies directed against PD-L1 that are useful in the prevention of pulmonary vascular remodeling and treatment of vascular conditions.

Pulmonary hypertension is a rare but severe condition characterized by high blood pressure in the lungs. Pulmonary arterial hypertension is a subset of PH in which the high blood pressure is localized to pulmonary arteries. In PAH, the arteries become narrowed and thickened which makes it difficult for the heart to pump. The heart can then become enlarged and weakened, which can cause other diseases such as right heart failure. A substantial obstacle in advancing the field of pulmonary hypertension, a disease for which there is currently no cure, is the lack of a well-understood mechanism for the development of the disease in many patients.

Interstitial lung disease (ILD), a collection of more than 200 lung disorders that involves the interstitium (the tissue and space around the air sacs of the lungs) and includes pulmonary fibrosis, is one of the most common causes for pulmonary hypertension. Pulmonary fibrosis refers to scarring of lung tissue. Pulmonary fibrosis may cause pulmonary hypertension when scarred lung tissue leads to compression of the vessels. The scar tissue increases resistance to blood flow from the heart to the lungs, leading to increased high pressure in the pulmonary arteries and the right heart ventricle. Idiopathic pulmonary fibrosis (IPF) is the most common type of pulmonary fibrosis. The modifier “idiopathic” signifies a disease with no known cause.

A current staple of treatment for PH is phosphodiesterase-5 inhbitors such as sildenafil, which is known to decrease MDSC arginase 1 (Arg1) and inducible nitric oxide synthase (iNOS) expression, leading to decreased immunosuppressive capabilities and increased CD8⁺ T cell activation. However, Group 3 PH patients have no disease-specific therapy to date, despite having a worse prognosis than demographically similar populations with advanced malignancy. Severity of PH is assessed by invasive measurement of mean pulmonary arterial pressure (mPAP). It is known that dysregulated myelopoiesis in response to chronic inflammatory injury can lead to an exhausted cellular phenotype with long-term immunosuppression, vascular injury (Boettcher et al., 2014), decreased functional status (Loftus, Mohr & Moldawer, 2018), and uncontrolled cellular proliferation (Strauss, 2015). Relatedly, granulopoiesis and subsequent neutrophilia is known to be an independent risk factor for early death in IPF (Kinder, Brown, Schwarz, Ix, Kervitsky & King, 2008); likewise, granulocytes have been implicated as pathogenic in a number of pulmonary vascular diseases (Taylor, Dirir, Zamanian, Rabinovitch & Thompson, 2018). As a point of comparison to the cancer literature, this may be due in part to an increase in circulating polymorphonuclear myeloid-derived suppressor cell (PMN-MDSC) as well as a corresponding elevation in PD-L1 expression by a neutrophilic subset of immature myeloid cells (Ballbach et al., 2017). Such a cell autonomous effect has been demonstrated in related disease models, whereby macrophage/monocyte subsets from patients with coronary artery disease express increase PD-L1, leading to T cell inhibition and a predisposition to reactivation of viral infection (Watanabe et al., 2017).

Immune checkpoints are regulators for maintaining systemic immune homeostasis and self-tolerance. Among them, the PD-1 pathway is utilized by cancer cells to escape the surveillance of the immune system. The use of myeloid cell-directed immune checkpoint inhibitors, such as PD-1 inhibitors, in cancer therapeutic regimens have proven a windfall in the treatment of many malignancies previously felt to be terminal. The specific immune checkpoint signaling axis of PD-1 and PD-L1 has been implicated in the pathogenesis of pulmonary fibrosis (Celada et al., 2018; Geng et al., 2019).

It has been suggested that the PDL-1 pathway confers protection against PH pathogenesis, as the blocking of PD-L1 expression on T regulatory cells (Tregs) was shown to abrogate Treg-mediated protection against PH following pulmonary vascular injury in rats (Tamosiuniene et al., Circ. Res. 2018).

Contrary to the findings of Tamosiuniene et al., the present disclosure describes a positive association between PD-L1 inhibition and reversal of PH symptoms. The anti-PD-L1 therapies described herein confer partial, and in some cases complete, reversal of vascular remodeling in a model of pulmonary vascular disease.

Accordingly, in some aspects, the present disclosure provides methods of treating pulmonary hypertension comprising administering a composition comprising a PD-L1 inhibitor or PD-1 inhibitor to a subject having pulmonary hypertension. In some embodiments, the subject has pulmonary arterial hypertension. In some aspects, the subject suffers from interstitial lung disease.

The PD-L1 inhibitor or PD-1 inhibitor of the disclosed methods may comprise a monoclonal antibody. Exemplary monoclonal antibodies include, but are not limited to, pembrolizumab, atezolizumab, avelumab, nivolumab, and durvalumab. In some embodiments, the monoclonal antibody is administered semi-weekly. In other embodiments, the antibody is administered every three weeks.

In some embodiments, the method further comprises the step of contacting myeloid-derived suppressor cells (MDSC) of the lung of the subject with the PD-L1 inhibitor or PD-1 inhibitor. In some embodiments, the method further comprises the step of diagnosing the subject as having pulmonary hypertension.

In some embodiments, the method further comprises administering an inhibitor of C-X-C chemokine receptor type 2 (CXCR2), also known as interleukin 8 receptor, beta (IL8RB). CXCR2 is a G-protein coupled receptor that binds to IL8 with high affinity.

In some embodiments, the subject may have been previously been treated for pulmonary hypertension. The subject may be a mammal, such as a human. The PD-L1 inhibitor or PD-1 inhibitor may be administered in a pharmaceutical composition comprising one or more additional pharmaceutically acceptable agents.

In other aspects, the present disclosure provides diagnostic methods. The diagnostic methods may be designed for identifying a subject having pulmonary hypertension that is sensitive to treatment with a PD-L1 inhibitor or a PD-1 inhibitor, the method comprising: (a) counting the absolute number of polymorphonuclear MDSC (PMN-MDSC) in a first biological sample obtained from a subject having a pulmonary hypertension; (b) administering a PD-L1 inhibitor or a PD-1 inhibitor to the subject; (c) counting the absolute number of PMN-MDSC in a second biological sample obtained from the subject; and (d) identifying the subject as having pulmonary hypertension that is sensitive to treatment with a PD-L1 inhibitor or a PD-1 inhibitor if the absolute number generated in (c) is not substantially lower than the absolute number generated in (a).

In some embodiments, steps (a) and (c) comprise counting the number of PMN-MDSC in samples obtained from the lung of a subject. In other embodiments, steps (a) and (c) comprise counting the number of PMN-MDSC in peripheral blood samples obtained from a subject.

In some embodiments, the diagnostic methods further comprise the step of administering to the subject a pharmaceutical composition comprising a PD-L1 inhibitor or PD-1 inhibitor.

Also provided herein are diagnostic methods for identifying a subject having a pulmonary hypertension that is sensitive to treatment with a CXCR2 inhibitor.

The details of one or more embodiments of the invention are set forth in the accompanying Figures, the Detailed Description, and the Examples. Other features, objects, and advantages of the invention will be apparent from the description and the claims.

### BRIEF DESCRIPTION OF THE DRAWINGS

The present invention is further described below with reference to the following non-limiting examples and with reference to the following figures, in which:

FIGS. 1A-1C show that Diphtheria Toxin (DT) administration to LysM.Cre-DTR (“mDTR”) mice results in a decrease in CD11c⁺ cells in the lung and spleen of mice given bleomycin. FIG. 1A is a schematic demonstrating the protocol for stimulation of myelopoiesis, with harvest occurring at day 33 (D33), after initiation of bleomycin protocol. FIG. 1B shows that injection (100 ng, intraperitoneal, twice weekly) of DT resulted in an absolute and relative (FIG. 1C; representative flow cytometric plots) decrease in the amount of CD11c⁺ cells within the lung and spleen of vehicle (Veh) or bleomycin (Blm)-stimulated mDTR mice. n=5-7 mice/group. In FIG. 1C, the % of cells negative and % of cells positive for DT is indicated in the top left and right corner of each panel, respectively. All data are presented as mean±SEM. P<0.05 was considered significant.

FIGS. 2A-2E show that Diphtheria Toxin (DT) administration to LysM.Cre-DTR (“mDTR”) mice given bleomycin results in an increase in pulmonary hypertension. FIG. 2A shows measurement of right ventricular systolic pressure (RVSP; mmHg) in mDTR mice given vehicle (Veh) or bleomycin (Blm) with or without DT. FIG. 2B shows th right ventricle to left ventricle plus septal mass ratio (RV:LV+S; %) between groups. FIG. 2C shows the absolute muscularized vessel counts per high-powered field (HPF). FIG. 2D shows the ratio of complete-to-partially muscularized vessels, as assessed by FIG. 2E. α-smooth muscle actin (α-sma; brown) immunohistochemical (IHC) stain of sectioned lungs from treatment groups as indicated. Scale bar 500 μm. n=5-9 mice/group. All data are presented as mean±SEM. P<0.05 was considered significant.

FIGS. 3A-3B show that Diphtheria Toxin (DT) administration to LysM.Cre-DTR (“mDTR”) mice given bleomycin results in no change in pulmonary fibrosis. FIG. 3A shows bibrosis scores as quantified between mDTR mice groups administered vehicle (Veh) or bleomycin (Blm), with or without DT. FIG. 3B shows the results of Masson's trichrome staining (MTC) of sectioned lungs from treatment groups. Scale bar 500 μm. n=5-7 mice/group. All data are presented as mean±SEM.

FIGS. 4A-4E show that LysM.Cre-DTR (“mDTR”) mice given diphtheria toxin (DT) and bleomycin display an increase in pulmonary myeloid-derived cells, characterized by an immunosuppressive signature. FIG. 4A shows representative flow cytometric plots displaying relative differences in CD11b⁺ cell population within lung and spleen, including pulmonary Ly6C^{hi}Ly6G⁻ and Ly6C^{lo}Ly6G⁺ cells, in mDTR mice with and without DT treatment, and vehicle or bleomycin. In FIG. 4A, the % of cells negative and % of cells positive for DT is indicated in the top left and right corner of each panel, respectively. FIGS. 4B-4C show absolute cell counts for treatment groups. FIGS. 4D-4E show arginase 1 (Arg1) expression by mean fluorescence intensity (MFI) in treated groups of pulmonary Ly6C^{hi}Ly6G⁻ and Ly6C^{lo}Ly6G⁺ cells, with representative histogram. n=5-9 mice/group. All data are presented as mean±SEM. P<0.05 was considered significant.

FIGS. 5A-5C demonstrate that myeloid cells isolated from LysM.Cre-DTR (“mDTR”) mice given diphtheria toxin (DT) suppress T cell proliferation, characteristic of myeloid-derived suppressor cells (MDSC). FIGS. 5A-5C show representative histograms and proliferation index (CD4+ and CD8+ cells, as % of T cells) after MDSC purified from spleens of mDTR mice treated with and without DT were cultured with T cells labeled with CTV, and stimulated with anti-CD3/CD28 antibodies at listed ratios. n=5 mice/group. All data are presented as mean±SEM. P<0.05 was considered significant.

FIGS. 6A-6D show that LysM.Cre-DTR (“mDTR”) mice given diphtheria toxin (DT) have increased pulmonary expression of PD-1 in T cells, and PD-L1 in myeloid-derived suppressor cell (MDSC). FIG. 6A shows representative flow plots and histograms detailing CD4+ and Treg cell—with relative abundance per CD4+ cells (%)—expression of PD-1 by mean fluorescence intensity (MFI) upon treatment of mDTR mice with or without DT in combination with vehicle (Veh) or bleomycin (Blm). In FIG. 6A, the % of cells negative and % of cells positive for DT is indicated in the left and right panels, respectively. FIG. 6B shows representative histograms of PD-L1 expression in MDSC sub-populations (monocytic, Mo-, and polymorphonuclear, PMN-MDSC) within exposed treatment groups. FIGS. 6C-6D shows the quantification of expression data expressed in sub-panels A and B, respectively. n=5-9 mice/group. All data are presented as mean±SEM. P<0.05 was considered significant.

FIGS. 7A-7F show that anti-PD-L1 treatment protects against development of pulmonary hypertension, but not pulmonary fibrosis, after induction of emergency myelopoiesis in the bleomycin (Blm) model. FIG. 7A shows illustrative right ventricular systolic pressure (RVSP)-time tracing in diphtheria toxin (DT) treated LysM.Cre-DTR (“mDTR”) mice exposed to vehicle (Veh) or bleomycin (Blm), treated with anti-PD-L1 antibody or IgG control. FIG. 7B shows the RVSP (mmHg) score and FIG. 7C shows the fibrosis score in the antibody-treated and control mDTR groups. FIG. 7D shows the results of MTC staining of lung sections from treatment groups. FIG. 7E shows the complete-to-partially muscularized pulmonary vessel ratio, assessed by FIG. 7F. α-smooth muscle actin (α-sma; brown) stain of lung sections. Scale bars 100 μm. n=5-7 mice/group. All data are presented as mean±SEM. P<0.05 was considered significant.

FIGS. 8A-8D show that anti-PD-L1 treatment results in a decrease in myeloid-derived suppressor cell (MDSC) within the lung, and improvement in markers of Treg exhaustion upon induction of emergency myelopoiesis in the bleomycin model. FIG. 8A show absolute CD11b⁺ and polymorphonuclear-MDSC (PMN-MDSC) counts after treatment with either IgG control or anti-PD-L1 antibody in vehicle (Veh) or bleomycin (Blm) exposed LysM.Cre-DTR (“mDTR”) mice given diphtheria toxin (DT). FIG. 8B show levels of expression of FoxP3 and interleukin-10 (IL-10) in pulmonary Treg cells of the antibody-treated and control mDTR groups, with FIG. 8C illustrating the representative histograms. FIG. 8D shows the percentage of CD62L+ cells (of CD8+ T cells) in both treatment groups. n=5-7 mice/group. All data are presented as mean±SEM. P<0.05 was considered significant.

FIGS. 9A-9C show that patients with interstitial lung disease (ILD) and pulmonary hypertension (PH) have increased expression of PD-L1 (CD274) on MDSC. FIG. 9A shows levels of expression of CD274 (PD-L1) in mean fluorescence intensity (MFI) by CD11b⁺CD33+HLA⁻DR⁻CD14⁻CD15⁺ cells (PMN-MDSC) in HC (n=8) and ILD with (n=15), and without PH (n=5) patient peripheral blood samples. FIG. 9B shows the percentage of CD274⁺CXCR2⁺ cells (of PMN-MDSC) between HC and ILD groups. FIG. 9C is a high-level schematic figure illustrating a mechanism of PD-1 action. All data are presented as median±IQR. P<0.05 was considered significant.

FIGS. 10A-10F show soluble growth factors and cytokines, analyzed by luminex assay, resulted from whole lungs of LysM.Cre-DTR (“mDTR”) mice treated either with or without diphtheria toxin (DT) and vehicle (Veh) or bleomycin (Blm) including: G-CSF (FIG. 10A), Eotaxin (FIG. 10B), IL-1β (FIG. 10C), IL-2 (FIG. 10D), MIP-2 (FIG. 10E), and RANTES (FIG. 10F). n=4-7 mice/group. All data are presented as mean±SEM. P<0.05 was considered significant.

FIG. 11 is a table containing a list of exemplary antibodies targeting the PD-1 pathway.

FIG. 12 is a schematic showing the study design of an exemplary clinical trial, e.g., the Myeloid cell targeting in Interstitial lung disease (ILD) associated Circulatory pathology via Immune Checkpint inhibition Trial (“MIC Check” trial). Abbreviations: ILD=Interstitial lung disease; PH=pulmonary hypertension; MDSC=myeloid-derived suppressor cell; RHC=right heart catheterization; PFT=pulmonary function test; HRCT=high-resolution CT; CT-ILD=connective tissue disease associated ILD; PBMC=peripheral blood mononuclear cell; IPAF=interstitial pneumonia with autoimmune features.

### DETAILED DESCRIPTION OF THE INVENTION

The present disclosure provides methods of treatment of pulmonary vascular diseases comprising administration of a PD-L1 inhibitor. In various embodiments, methods of treatment of PH (e.g., PAH) are provided. Further provided herein are diagnostic methods for assessing patient sensitivity to therapies. In various embodiments, methods of identifying a subject having a pulmonary hypertension that is sensitive to treatment with a PD-L1 inhibitor or a PD-1 inhibitor are provided. In various embodiments, subjects identified as having a PH that is sensitive to such treatment may be subsequently administered the PD-L1 inhibition treatment.

In some embodiments, the present disclosure provides methods of treating pulmonary hypertension comprising administering a composition comprising a PD-L1 inhibitor or PD-1 inhibitor to a subject having pulmonary hypertension. In some embodiments, the subject has pulmonary arterial hypertension. In some aspects, the subject suffers from interstitial lung disease.

In some embodiments, the disclosure provides methods of treating pulmonary hypertension comprising administering a composition consisting essentially of a PD-L1 inhibitor or PD-1 inhibitor to a subject having pulmonary hypertension, such as pulmonary arterial hypertension. In compositions consisting essentially of a PD-L1 inhibitor or PD-1 inhibitor, the PD-(L)1 inhibitor is the only active pharmaceutical ingredient of the composition. In other embodiments, the disclosure provides methods of treatment comprising administering a composition comprising a PD-(L)1 inhibitor and one or more additional active pharmaceutical agents, such as a vasodilatory agent. Also provided are methods of treatment comprising administering a PD-L1 inhibitor and a second (or third or fourth) active pharmaceutical ingredient, in separate compositions.

The PD-L1 inhibitor or PD-1 inhibitor may be administered in a dose of about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 20 mg/kg, 25 mg/kg, 35 mg/kg, or 50 mg/kg of subject. The PD-L1 inhibitor or PD-1 inhibitor may be administered in a dose of about 10 mg/kg of subject.

In other embodiments, the PD-L1 inhibitor or PD-1 inhibitor may be administered in a dose of about 200 mg to about 1200 mg. The PD-L1 inhibitor or PD-1 inhibitor may be administered in a dose of about 50 mg, about 75 mg, about 100 mg, about 125 mg, about 150 mg, about 175 mg, about 200 mg, about 225 mg, about 250 mg, about 300 mg, about 400 mg, about 500 mg, about 600 mg, about 700 mg, about 800 mg, about 900 mg, about 1000 mg, about 1100 mg, about 1150 mg, about 1200 mg, about 1250 mg, about 1300 mg, about 1400 mg, or about 1500 mg.

Pulmonary hypertension—defined as mean pulmonary artery pressure (mPAP) greater than 25 mmHg—is a heterogenous disease entity encompassing a broad patient population. Although prognosis has improved incrementally over the last several decades, outcomes continue to be universally grim for those diagnosed with PH, with five-year survival rates worse than that of many malignancies (McGoon et al., 2013). Moreover, while effective vasodilator-based therapies have resulted in improved morbidity (Taichman et al., 2013), curative treatment remains elusive. Therefore, novel perspectives on disease pathogenesis are required. A promising area of investigation in PH research involves the role myeloid cells play in pulmonary vascular remodeling.

Immature myeloid cells that are in a pathologic state of activation have been identified in many inflammatory disorders, including pulmonary arterial hypertension. Myeloid-derived suppressor cells (MDSC) are a subtype of immature myeloid cells that were initially described in relation to the immune escape and proliferation of certain cancers. It is known that peripheral blood MDSC are elevated in pediatric patients with pulmonary hypertension secondary to congenital heart disease compared to healthy controls, especially in female patients. In addition, in several murine models of disease, polymorphonuclear MDSC (PMN-MDSC), a subpopulation of MDSC, promote PH, with a reduction in PMN-MDSC trafficking to the lung attenuating pulmonary vascular remodeling. Despite these findings, a profile of MDSC, including potentially important differences in PMN-MDSC and monocytic MDSC (Mo-MDSC) sub-groups, is unknown in adult patients with either familial or idiopathic PAH.

Whereas a variety of bone marrow-derived cells are believed to have autonomous effects on development of PH (Farha et al., 2011; Asosingh et al., 2012), a causative role for a specific sub-population of leukocytes—myeloid-derived suppressor cells has recently been discovered (Bryant et al., 2018). MDSC—precursor cells similar in gross appearance to either immature monocytes (Mo-MDSC) or neutrophils (PMN-MDSC)—developed evolutionarily to protect the host organism from infection and assist in wound repair, primarily acting to regulate effector T cell response to injury (Gabrilovich, 2017).

Furthermore, pathologic activation of MDSC has been demonstrated to correlate with severity of PH (Yeager et al., 2012), providing a potential missing link between myeloid-cell proliferative response and T-cell dysfunction previously well-described in the progression of pulmonary vasculopathy (Huertas et al., 2016). Significantly, MDSC induce T-cell exhaustion and senescence in large part through expression of immune checkpoint proteins including programmed death-ligand 1 (PD-L1; also known as CD274), which acts through binding of the protein's canonical receptor programmed cell death protein 1 (PD-1; also known as CD279) on neighboring lymphocytes. Application of immune checkpoint inhibitors, therapeutic antibodies with specificity for blocking either the receptor or the ligand (PD-1 or PD-L1, respectively), have resulted in astonishing progress in the treatment of primarily solid organ and hematologic cancers over the past decade.

Studies on PD-L1 in pulmonary hypertension development have previously focused on the role of effector T cells and endothelial cells, whereas little has been known about the role of MDSCs. The combination of MDSC targeting with immune checkpoint inhibitor treatment has been applied effectively to several preclinical tumor models and cancer patients. An example of this combinatory approach relevant to pulmonary hypertension research is the use of PD-1 blockade with phenformin, an antidiabetic drug from the biguanide class. In one study, phenformin was able to enhance the effect of immune checkpoint inhibition, as evidenced by an increase in CD8⁺ T cell infiltration in a melanoma model (Kim et al., 2017).

Previously, it was indicated that PD-L1 surface expression is elevated on circulating MDSCs from patients with PAH. Reference is made to Bryant et al., A checkpoint on innate myeloid cells in pulmonary arterial hypertension. Putin. Circ. 9(1): 1-5 (2019), herein incorporated by reference. It was shown that the ratio of cells with PMN-MDSC surface markers (CD14⁻CD15⁺) were elevated in a cohort of human patients with PAH, while Mo-MDSCs (CD14⁺) were decreased. It was found that the population of PD-L1-expressing MDSC was higher in patients with PAH compared to controls; in addition, the surface expression of PD-L1 was higher in both of the MDSC sub-populations of PAH patients compared to healthy controls. Although no significant correlation was observed between PD-L1 expression on Mo-MDSC and mPAP, there was a significant positive correlation between PMN-MDSC expression of PD-L1 with severity of PH in the patients. This clinical data suggested that PD-1/PD-L1 plays a role in development of PH. In addition, this study showed a significant elevation of PD-L1 expression on MDSC in hypoxia-treated mice.

The findings in Bryant et al., 2019 suggested that MDSC, in combination with PD-L1 expression, may mediate inflammation related to chronic vasculopathy. Additionally, in view of the role chemokine receptors such as CXCR2 are known to play in the pathogenesis of PAH (Burton et al, 2011, incorporated herein by reference) and the potential for synergism between these receptors and PD-1/PD-L1 expression (Highfill et al., 2014), their analysis in circulating subsets of MDSC deserve more attention as putative inflammatory markers of disease.

Blockade of PD-1 by antibodies can enhance the immune response to cancerous cells in the patient. The interaction between PD-1 and PD-L1 results in a decrease in tumor infiltrating lymphocytes, a decrease in T-cell receptor mediated proliferation, and immune evasion by the cancerous cells (Dong et al. (2003) J Mol Med 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 to PD-L1 and the effect is additive when the interaction of PD-1 to PD-L2 is blocked as well (Iwai et al. (2002) PNAS 99:12293-7; Brown et al. (2003) J. Immunol. 170:1257-66). In some aspecta, the present invention relates to treatment of a subject in vivo using an anti-PD-1 antibody such that harmful pulmonary vascularization is inhibited. An anti-PD-1 antibody may be used alone to inhibit pulmonary vascularization. Alternatively, an anti-PD-1 antibody may be used in conjunction with other immunogenic agents, standard cancer treatments, or other antibodies.

The methods provided herein of treating pulmonary hypertension may comprise administering a PD-L1 inhibitor or PD-1 inhibitor in combination with another active ingredient. In some embodiments, the methods provided herein comprise administering the inhibitor in the absence of any other active ingredient, such as a phosphodiesterase-5 inhibitor. Accordingly, in some embodiments, of the methods provided herein, the method does not comprise a step of administering a phosphodiesterase-5 inhibitor. In some embodiments, the PD-L1 inhibitor or PD-1 inhibitor does not comprise a monoclonal antibody.

### Monoclonal Antibody PD-L1 and PD-1 Inhibitors

The presently disclosed methods of treatment and diagnostic methods may be used with any PD-1 inhibitor or PD-L1 inhibitor. The inhibitors of the disclosure may be FDA-approved for use in humans. The inhibitors may comprise one or more monoclonal antibodies.

The PD-1 and PD-L1 inhibitors of the disclosure may comprise human antibodies. In other embodiments, the inhibitors may comprise chimeric or humanized antibodies. The inhibitors may comprise natural or recombinant antibodies. In particular embodiments, the inhibitors may comprise recombinant human antibodies. The inhibitors may otherwise comprise recombinant chimeric antibodies. The inhibitors may otherwise comprise recombinant humanized antibodies.

In some embodiments, the PD-1 and PD-L1 inhibitors comprise monoclonal antibodies that are fucosylated. In other embodiments, the inhibitors comprise antibodies that are not fucosylated.

The PD-1 and PD-L1 inhibitors of the disclosure comprise antibodies with affinity to PD-1 or PD-L1. The PD-1 and PD-L1 inhibitors may comprise antibodies with high affinity to PD-1 or PD-L1. The inhibitors may comprise affinity-maturated antibodies.

The present disclosure embraces bispecific antibodies comprising at least one first binding specificity for PD-1 and a second binding specificity for a second target epitope. In a particular embodiment of the invention, the second target epitope is an Fc receptor, e.g., human FcγRI (CD64) or a human Fcα receptor (CD89). Therefore, the invention includes bispecific molecules capable of binding both to FcγR or FcαR expressing effector cells (e.g., monocytes, macrophages or polymorphonuclear cells (PMNs)), and to target cells expressing PD-1. These bispecific molecules target PD-1 expressing cells to effector cell and trigger Fc receptor-mediated effector cell activities, such as phagocytosis of an PD-1 expressing cells, antibody dependent cell-mediated cytotoxicity (ADCC), cytokine release, or generation of superoxide anion.

The term “monoclonal antibody,” as used herein, refers to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, the term “subject” includes any human or nonhuman animal. The term “nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the T-cell mediated immune response. In a particular embodiment, the methods are particularly suitable for treatment of cancer cells in vivo. To achieve antigen-specific enhancement of immunity, the anti-PD-1 antibodies can be administered together with an antigen of interest. When antibodies to PD-1 are administered together with another agent, the two can be administered in either order or simultaneously.

As used herein, an antibody that “binds to PD-F” is intended to refer to an antibody that binds to PD-1 with a dissociation constant (K_{D}) of 1×10⁻⁷ M or less, more preferably 5×10⁻⁸ M or less, more preferably 1×10⁻⁸ M or less, more preferably 5×10⁻⁹ M or less. In particular, the antibodies of the disclosure bind to human PD-1 with a dissociation constant (K_{D}) of 1×10⁻⁷ M or less, more preferably 5×10⁻⁸ M or less, more preferably 1×10⁻⁸ M or less, more preferably 5×10⁻⁹ M or less. Standard assays to evaluate the binding affinity of the antibodies toward PD-1 are known in the art, including for example, ELISAs, Western blots and RIAs. The binding kinetics (e.g., binding affinity) of the antibodies also can be assessed by standard assays known in the art, such as by Biacore analysis.

In some embodiments, the antibody of the disclosure may block binding of PD-L1 (e.g., human PD-L1) to PD-1 (e.g., human PD-1) with an IC₅₀ of about 1 nM or lower. The blockade of ligand binding can be measured and the IC₅₀ calculated using any method known in the art, for example, FACS or FMAT methods.

The invention also comprises an antibody that competes for a binding epitope on human PD-1 with any of the antibodies described above, and which blocks the binding of human PD-L1 to human PD-1 with an IC₅₀ of about 1 nM or lower.

Exemplary PD-1 inhibitors useful in the present disclosure include monoclonal antibodies that target the PD-1 signaling pathway. PD-1 inhibitors may comprise pembrolizumab, atezolizumab, avelumab, nivolumab, and/or durvalumab. In other embodiments, the PD-1 inhibitor may comprise pidilizumab (CT-011) or MPDL3280A. The monoclonal antibodies may be approved for use in humans by the FDA, EMA or a similar regulatory agency.

As used herein, the term “pembrolizumab” embraces Keytruda®, an FDA-approved PD-1 inhibitor. The term also embraces antibodies known as MK-3475, lambrolizumab, and any biosimilar or generic versions of Keytruda, pembrolizumab and lambrolizumab.

Pembrolizumab is described in U.S. Pat. Nos. 8,354,509, 8,900,587 and 8,168,757, each of which are incorporated herein by reference.

As used herein, the term “atezolizumab” embraces Tecentriq®, an FDA-approved PD-1 inhibitor. The term also embraces antibodies known as MPDL3280A, RO5541267 and any biosimilar or generic versions of Tecentriq and atezolizumab.

Atezolizumab is described in U.S. Pat. No. 8,217,149, US Patent Publication No. 2016/62405188, and International Patent Publication No. WO 2014/151006, each of which are incorporated herein by reference.

As used herein, the term “avelumab” embraces Bavencio®, an FDA-approved PD-1 inhibitor. The term also embraces antibodies known as MSB0010718C and any biosimilar or generic versions of Bavencio and avelumab.

Avelumab is described in US Patent Publication No. 2017/0253653 and International Patent Publication Nos. WO 2018/065938 and WO 2018/162446, each of which are incorporated herein by reference.

As used herein, the term “nivolumab” embraces Opdivo®, an FDA-approved PD-1 inhibitor. The term also embraces antibodies known as BMS-936558, MDX-1106, and ONO-4538, and any biosimilar or generic versions of Opdivo and nivolumab.

Nivolumab is described in U.S. Pat. Nos. 9,402,899, 8,779,105, 9,073,994, 8,728,474 and 9,856,320 and US Patent Publication No. 2012/6164744, each of which are incorporated herein by reference.

In some embodiments, the monoclonal antibody does not comprise nivolumab, pembrolizumab, or atezolizumab. In some embodiments, the antibody comprises durvalumab.

As used herein, the term “durvalumab” embraces Imfinzi®, an FDA-approved PD-1 inhibitor. The term also embraces antibodies known as MEDI4736 and any biosimilar or generic versions of Opdivo and durvalumab.

Durvalumab is described in U.S. Pat. Nos. 8,779,108 and 10,336,823, each of which are incorporated herein by reference.

The antibodies of the disclosure may be, for example, full-length anti-PD1 and anti-PDL1 antibodies, for example of an IgG1 or IgG4 isotype. Alternatively, the antibodies may be antibody fragments, such as Fab or Fab′2 fragments, or single chain antibodies.

The disclosure also provides an immunoconjugate comprising an antibody of the invention, or antigen-binding portion thereof, linked to a therapeutic agent, such as a cytotoxin or a radioactive isotope. The invention also provides a bispecific molecule comprising an antibody, or antigen-binding portion thereof, of the invention, linked to a second functional moiety having a different binding specificity than said antibody, or antigen-binding portion thereof.

Compositions comprising an anti-PD1 or anti-PD-L1 antibody, or antigen-binding portion thereof, or immunoconjugate or bispecific molecule of the invention, and a pharmaceutically acceptable agent, are also provided.

The antibodies of the invention are characterized by particular functional features or properties of the antibodies. For example, the antibodies bind specifically to PD-1 (e.g., bind to human PD-1 and may cross-react with PD-1 from other species, such as cynomolgus monkey). Preferably, an antibody of the invention binds to PD-1 with high affinity, for example with a K_{D} of 1×10⁻⁷M or less. The anti-PD-1 antibodies of the invention may exhibit one or more of the following characteristics:

(a) binds to human PD-1 with a K_{D} of 1×10⁻⁷M or less;

(b) does not substantially bind to human CD28, CTLA-4 or ICOS;

(c) increases T-cell proliferation in an Mixed Lymphocyte Reaction (MLR) assay;

(d) increases interferon-gamma production in an MLR assay;

(e) increases IL-2 secretion in an MLR assay;

(f) binds to human PD-1;

(g) inhibits the binding of PD-L1 to PD-1;

(h) stimulates antigen-specific memory responses; and

(i) stimulates antibody responses.

An antibody of the disclosure may exhibit any combination of the above-listed features, such as two, three, four, five or more of the above-listed features.

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

### Pharmaceutical Compositions and Methods of Administration

In another aspect, the present invention provides a composition, e.g., a pharmaceutical composition, containing one or a combination of monoclonal antibodies, or antigen-binding portion(s) thereof, of the present invention, formulated together with a pharmaceutically acceptable agent. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e., combined with other agents. For example, the combination therapy can include an anti-PD-1 antibody of the present invention combined with at least one other anti-inflammatory or immunosuppressant agent. Examples of therapeutic agents that can be used in combination therapy are described in greater detail below in the section on uses of the antibodies of the invention.

As used herein, “pharmaceutically acceptable agent” includes any and all carriers, buffers, solvents, dispersion media, coatings, antibacterial and antifungal agents, isotonic and absorption delaying agents, and the like that are physiologically compatible. Preferably, the agent is suitable for intravenous, intramuscular, subcutaneous, parenteral, spinal or epidermal administration (e.g., by injection or infusion). Depending on the route of administration, the antibody may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable agents include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable agent.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the PD-1 or PD-L1 inhibitor, a preferred dose protocol is one involving the maximal dose or dose frequency that avoids significant undesirable side effects. The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Exemplary dosage regimens for an anti-PD-1 antibody of the invention include 10 mg/kg or 1 mg/kg body weight via intravenous administration, with the antibody being given using one of the following dosing schedules: (i) every four weeks for six dosages, then every three months; (ii) every three weeks; (iii) 10 mg/kg body weight once followed by 1 mg/kg body weight every three weeks. In other embodiments, a total weekly dose may comprise at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, or at least 50 mg/kg.

In other embodiments, a daily dosage might range from about 0.0001 mg/kg, 0.001 mg/kg, 0.01 mg/kg, 0.1 mg/kg, 1 mg/kg, 10 mg/kg to up to 100 mg/kg, 1000 mg/kg, 10000 mg/kg or more, of the patient's body weight depending on the factors mentioned above. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the subject's body weight.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Doses of antibodies of the invention may be repeated and the administrations may be separated by at least 1 day, 2 days, 3 days, 5 days, 10 days, 15 days, 30 days, 45 days, 2 months, 75 days, 3 months, or at least 6 months.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-PD-1 antibody of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means. Preferred routes of administration for antibodies of the invention include intravenous, intramuscular, intradermal, intraperitoneal, subcutaneous, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. The disclosure of these patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The anti-PD-1 antibodies of the disclosure can be co-administered with one or other more therapeutic agents, e.g., an immunosuppressive agent or a vasodilatory agent. The antibody can be linked to the agent (as an immunocomplex) or can be administered separate from the agent. In the latter case (separate administration), the antibody can be administered before, after or concurrently with the agent or can be co-administered with other known therapies.

Also within the scope of the present disclosure are kits comprising the antibody compositions of the invention (e.g., human antibodies) and instructions for use. The kit can further contain a least one additional reagent, or one or more additional human antibodies of the invention (e.g., a human antibody having a complementary activity which binds to an epitope in PD-1 antigen distinct from the first human antibody). Kits typically include a label indicating the intended use of the contents of the kit. The term label includes any writing, or recorded material supplied on or with the kit, or which otherwise accompanies the kit.

### C-X-C Motif Chemokine Receptor Type 2 (CXCR2) Inhibitors

An association between decreased MDSC homing to the inflamed lung and attenuation of pulmonary vascular remodeling with administration of a selective CXCR2 inhibitor has been demonstrated. Additional supportive data, generated primarily from the cancer and autoimmunity literature, establishes a role for CXCR2-mediated trafficking and activation in PMN-MDSC-mediated pathology. For example, in an inflammatory colitis and colon cancer model, CXCR2 null mice were protected against development of disease, with tumor progression restored only after adoptive transfer of activated MDSCs. In another model examining pancreatic cancer, it appeared that targeting CXCR2 expression by immune cells was protective against malignant progression and metastasis, primarily through promotion of effector T lymphocyte—CD8⁺ Tcell—activity. And at least one study has demonstrated a protective role for CXCR2 overexpression in endothelial cells in the monocrotaline rat model, with marked decrease in neutrophil accumulation to the lung and decreases in interleukin-8 (IL-8) expression within the lung.

Although CXCR2 is expressed highly by circulating neutrophils in relevant models of comparison, such as cancer, it is primarily and functionally associated with upregulation on PMN-MDSCs. Additionally, other cell-surface chemokine receptors commonly expressed by Mo-MDSC, such as CCR2 and CX3CR1, are highly expressed in circulating immune cells accumulating within the lungs of mice with chronic hypoxia-induced pulmonary hypertension.

It was previously shown that granulocytic MDSCs (G-MDSCs) expressing CXCR2 can cause PH in a model of pulmonary fibrosis. Reference is made to Bryant et al., 2018, incorporated herein by reference. In addition, it was demonstrated human pathology consistent with preclinical studies in patients with IPF. Murine pulmonary vascular disease was completely attenuated after administering antagonists of CXCR2-mediated trafficking of G-MDSCs.

CXCR2 antagonists are currently being studied in patients with severe chronic obstructive pulmonary disease, having been proven to be both safe and efficacious in phase II clinical trials. Bryant et al., 2018 provides preclinical evidence that CXCR2 inhibitors may be used to prevent and potentially treat PH associated with IPF.

Acting in a synergistic fashion with novel immunotherapeutic agents directed against PD-1, provided herein are CXCR2 inhibitors that may be able to impede MDSC movement and function. Exemplary CXCR2 inhibitors include, but are not limited to, AZD5059, reparixin, navarixin, danirixin and SX-682. Danirixin is disclosed, e.g., in Miller et al. Eur J Drug Metab Pharmacokinet (2014) 39: 173-181; and Miller et al., BMC Pharmacology and Toxicology (2015), 16: 18. Reparixin is disclosed, e.g., in Zarbock et al., British Journal of Pharmacology (2008), 1-8. Navarixin is disclosed, e.g., in Ning et al., Mol Cancer Ther. 2012; 11(6): 1353-64. SX-682 is disclosed, e.g. in Liao et al., Cancer Cell, 35(4):559-572 (2019).

### Diagnostic Methods

In some aspects, the present disclosure provides diagnostic methods. The diagnostic methods may be designed for identifying a subject having pulmonary hypertension that is sensitive to treatment with a PD-L1 inhibitor or a PD-1 inhibitor. The methods may comprise (a) counting the absolute number of polymorphonuclear MDSC (PMN-MDSC) in a first peripheral blood sample obtained from a subject having a pulmonary hypertension; (b) administering a PD-L1 inhibitor or a PD-1 inhibitor to the subject; (c) counting the absolute number of PMN-MDSC in a second peripheral blood sample obtained from the subject; and (d) identifying the subject as having pulmonary hypertension that is sensitive to treatment with a PD-L1 inhibitor or a PD-1 inhibitor if the absolute number generated in (c) is not substantially lower than the absolute number generated in (a). In certain embodiments, the peripheral blood sample is obtained from vessesls surrounding one or both lungs.

In other embodiments, the methods may comprise the following steps: (a) counting the absolute number of polymorphonuclear MDSC (PMN-MDSC) in a first biological sample obtained from the lung tissue of a subject having a pulmonary hypertension; (b) administering a PD-L1 inhibitor or a PD-1 inhibitor to the subject; (c) counting the absolute number of PMN-MDSC in a second biological sample obtained from the lung tissue of the subject; and (d) identifying the subject as having pulmonary hypertension that is sensitive to treatment with a PD-L1 inhibitor or a PD-1 inhibitor if the absolute number generated in (c) is not substantially lower than the absolute number generated in (a).

In some embodiments, the diagnostic methods further comprise the step of administering to the subject a pharmaceutical composition comprising a PD-L1 inhibitor or PD-1 inhibitor. In some embodiments, the PD-L1 inhibitor PD-1 inhibitor is a monoclonal antibody, e.g. pembrolizumab, atezolizumab, avelumab, nivolumab, and durvalumab. In some embodiments, the monoclonal antibody is administered semi-weekly. In other embodiments, the antibody is administered every three weeks.

The PD-L1 inhibitor or PD-1 inhibitor may be administered in a dose of about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 20 mg/kg, 25 mg/kg, 35 mg/kg, or 50 mg/kg of subject. The PD-L1 inhibitor or PD-1 inhibitor may be administered in a dose of about 10 mg/kg of subject. In other embodiments, the PD-L1 inhibitor or PD-1 inhibitor may be administered in a dose of about 200 mg to about 1200 mg. The PD-L1 inhibitor or PD-1 inhibitor may be administered in a dose of about 50 mg, about 75 mg, about 100 mg, about 125 mg, about 150 mg, about 175 mg, about 200 mg, about 225 mg, about 250 mg, about 300 mg, about 400 mg, about 500 mg, about 600 mg, about 700 mg, about 800 mg, about 900 mg, about 1000 mg, about 1100 mg, about 1150 mg, about 1200 mg, about 1250 mg, about 1300 mg, about 1400 mg, or about 1500 mg.

Also provided herein are diagnostic methods for identifying a subject having a pulmonary hypertension that is sensitive to treatment with a PD-L1 inhibitor or a PD-1 inhibitor, the method comprising: (a) detecting the expression levels of PD-1 receptor on the surface of PMN-MDSC in a biological sample obtained from a subject having a pulmonary hypertension; and (b) identifying the subject as having a pulmonary hypertension that is sensitive to treatment with a PD-L1 inhibitor or a PD-1 inhibitor if the expression levels are substantially lower than normal controls. Further provided herein are methods comprising: (a) detecting the counts of circulating PMN-MDSC in a biological sample obtained from a subject having a pulmonary hypertension; and (b) identifying the subject as having a pulmonary hypertension that is sensitive to treatment with a PD-L1 inhibitor or a PD-1 inhibitor if the counts are substantially higher than normal controls. In the disclosed methods in which PD-1 receptor expression levels are determined, biological samples may be obtained from peripheral blood or lung tissue of the subject.

As used herein, “normal controls” refers to subjects that do not have phenotypes associated with pulmonary hypertension and/or nearly undetectable PD-1/PD-L1 expression levels in the peripheral blood. Methods of measuring PD-1/PD-L1 expression levels in the peripheral blood are known in the art. For instance, a VENTANA® PD-L1 (SP263) assay may be used. Reference is made to https://diagnostics.roche.com/global/en/products/tests/ventana-pd-11-_sp263-assay2.html#productSpecs, which is incorporated herein by reference.

In some embodiments, these diagnostic methods further comprise the step of administering to the subject a pharmaceutical composition comprising a PD-L1 inhibitor or PD-1 inhibitor.

The above-described step of identifying subjects as having a pulmonary hypertension that is sensitive to treatment with a PD-L1 inhibitor or a PD-1 inhibitor may further comprise the administration of one or more tests designed to assess the patients' vascular condition, e.g. pulmonary vascular condition. In addition, the step of administering the pharmaceutical composition following performance of the diagnostic method may further comprise monitoring the patient for symptoms of pulmonary vascularization, PH or PAH, e.g. by administration of one ore more of these tests or assessments.

These tests or assessments include, but are not limited to, pulmonary function tests (PFTs), High Resolution Computed Tomography (HRCT), pulmonary vascular response by right heart catheterization (RHC), echocardiogram (ECHO), an autoimmune panel, comprehensive metabolic panel (CMP) test, complete blood count (CBC) test, 6-minute walk distance test (6MWD), a questionnaire, CBC/CMP, Brain natriuretic peptide (BNP) test, urinalysis (UA), peripheral blood mononuclear cells (PBMC) and serum analysis, or an immunophenotype screen based on absolute counts of circulating MDSC cells and PD-L1 expression levels on MDSC. In various embodiments, patients are assessed by pulmonary vascular response by right heart catheterization (RHC) to the anti-PD(L)1 therapy.

Also provided herein are diagnostic methods for identifying a subject having a pulmonary hypertension that is sensitive to treatment with a CXCR2 inhibitor, the method comprising: (a) detecting the expression levels of CXCR2 receptor on the surface of PMN-MDSC in a biological sample obtained from a subject having a pulmonary hypertension; and (b) identifying the subject as having a pulmonary hypertension that is sensitive to treatment with a CXCR2 inhibitor if the expression levels are substantially lower than normal controls. Biological samples may be obtained from peripheral blood or lung tissue of the subject. In some embodiments, these diagnostic methods further comprise the step of administering to the subject a pharmaceutical composition comprising a CXCR2 inhibitor.

The above-described steps of identifying subjects having a pulmonary hypertension that is sensitive to treatment with a CXCR2 inhibitor may further comprise the administration of one or more of the above-described tests designed to assess the patients' vascular condition, e.g. pulmonary vascular condition. In addition, the step of administering the pharmaceutical composition comprising a CXCR2 inhibitor following performance of the diagnostic method may further comprise monitoring the patient for symptoms of pulmonary vascularization, PH or PAH, e.g. by administration of one ore more of these tests or assessments.

The details of one or more embodiments of the invention are set forth in the accompanying Figures, the Detailed Description, and the Examples. Other features, objects, and advantages of the invention will be apparent from the description and the claims.

The Examples demonstrate, inter alia, that mice undergoing induction of emergency myelopoeises displayed more severe PH, right ventricular remodeling, and pulmonary vascular muscularization, relative to controls, without a change in lung fibrosis. The observed worsening of PH was associated with increased pulmonary myeloid-derived suppressor cell counts (MDSC), and in particular polymorphonuclear MDSC (PMN-MDSC). Treatment with anti-PDL1 antibodies normalized pulmonary pressures in mice.

PD-L1 expression was likewise found to be elevated on circulating PMN-MDSC from patients with interstitial lung disease and PH. The Examples provide for an evaluation of the inhibitory effects of anti-PDL1 antibodies on PH symptoms in human subjects and patients, based on successful studies of these inhibitors in mice.

### EXAMPLES

The invention is further illustrated by the following examples which are intended to illustrate but not limit the scope of the invention.

### Example 1

Emergency Myelopoiesis Contributes to Pulmonary Vascular Remodeling, and Pulmonary Hypertension, with No Alteration in Pulmonary Fibrosis

Emergency myelopoiesis is primarily an evolutionary conserved response to infectious disease, whereby immature bone marrow-derived cells replenish those mature leukocytes lost in combatting illness (Chiba et al., 2018). It has recently been demonstrated that emergency myelopoiesis can be induced in response to intraperitoneal macrophage apoptosis induced by clodronate liposome injections (Bryant et al., 2018), building upon prior work demonstrating an increase in granulopoiesis, specifically, in response to chronic clodronate liposome administration in a model of cardiac injury (van Amerongen, Harmsen, van Rooijen, Petersen & van Luyn, 2007). Intriguingly, cell-specific targeting of myeloid cells themselves—using a LysM.Cre-DTR, or “mDTR”, mouse model—has likewise been shown to elicit increased granulopoiesis with an expansion in circulating Gr-1⁺ cells after induction of cell death with diphtheria toxin (DT) (Goren et al., 2009). Therefore, it was of interest to examine whether induction of emergency myelopoiesis using the mDTR model would result in an increase in myeloid-derived cells in a model of pulmonary vascular disease.

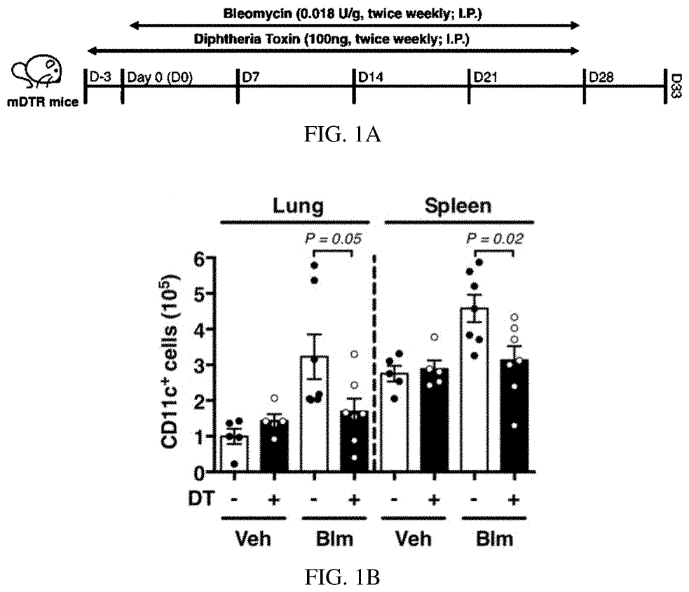

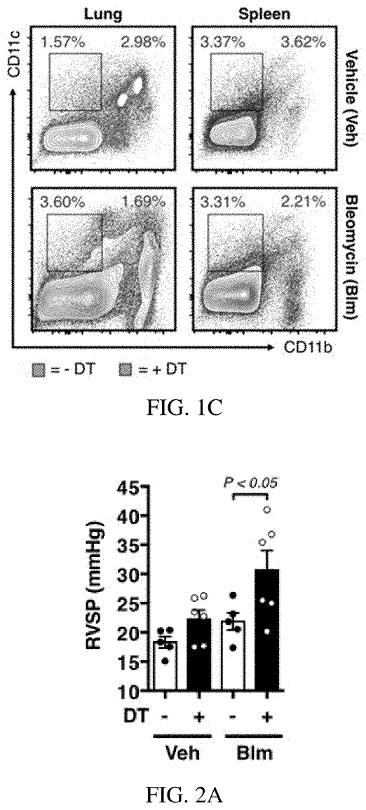

In order to test this hypothesis, bleomycin-associated pulmonary fibrosis and PH were stimulated in mDTR mice administered concurrently either vehicle or DT, in order to induce emergency myelopoiesis (FIG. 1A). First, however, it was of interest to confirm that, in a population of CD11b^{lo}CD11c⁺ cells within the lung and spleen, inclusive of macrophage subsets expected to undergo apoptosis, an expected reduction in cell numbers could be achieved upon DT administration. Such a reduction was successfully demonstrated in absolute cell numbers upon completion of the full DT injection protocol, but only significantly so in mice co-stimulated by the bleomycin-administration (FIGS. 1B and 1C).

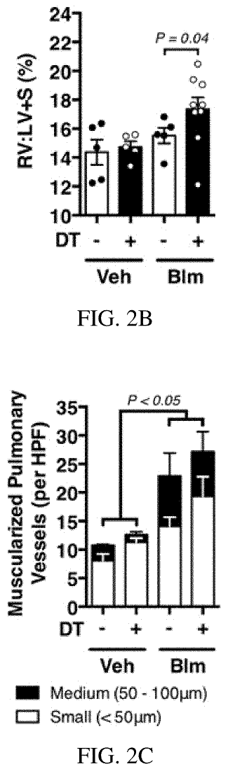

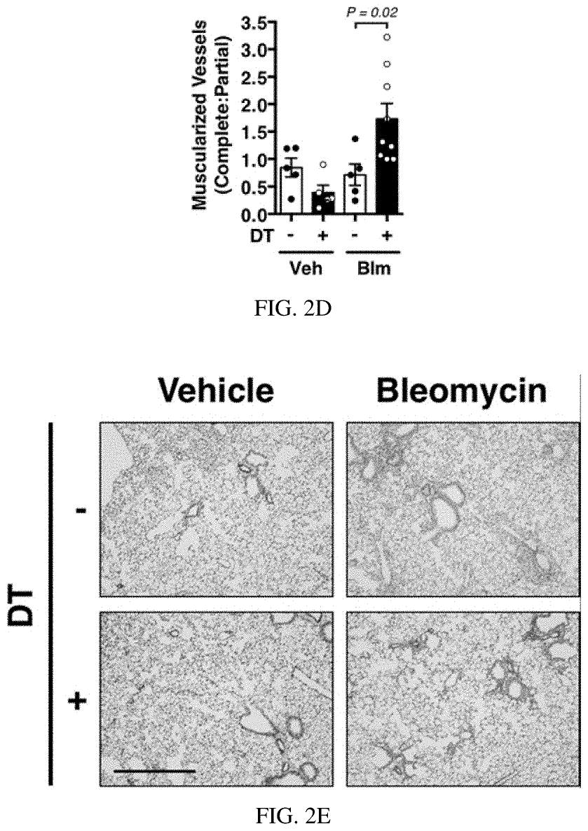

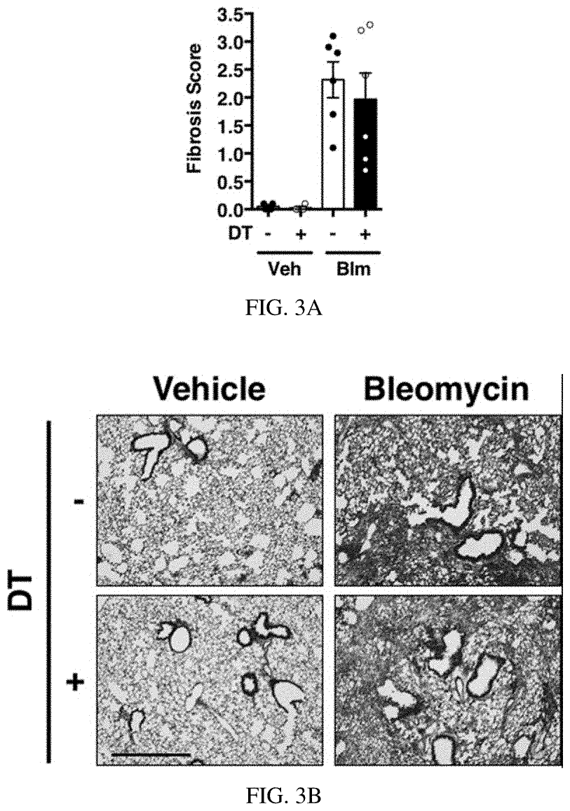

Next, right-ventricular systolic pressure (RVSP; mmHg) was measured in mDTR mice given bleomycin or vehicle. Consistent with prior data using the clodronate liposome model (Bryant et al., 2018; Pi et al., 2018), mDTR mice given bleomycin and DT displayed significantly higher RVSP than control mice (FIG. 2A), with an expected increase in right ventricular remodeling, as assessed by the right ventricle to left ventricle plus septal mass ratio (RV:LV+S; %) (FIG. 2B). Additionally, although there was no significant difference between bleomycin-treated groups in absolute small and medium sized pulmonary vessel muscularization (FIG. 2C), there was a significant increase in the ratio of fully to partially muscularized vessels (FIG. 2D), assessed by α-smooth muscle actin (α-sma) staining (FIG. 2E). Importantly, there was no significant difference in pulmonary fibrosis, as assessed by the modified Ashcroft score, between bleomycin-treated groups (FIGS. 3A and 3B). Thus, from these data it is concluded that selective cell depletion of myeloid cells, using the mDTR mouse model, results in worsened pulmonary vascular remodeling that cannot be attributed to major differences in lung fibrosis and vessel drop-out.

### Emergency Myelopoiesis Stimulates Myeloid-Derived Suppressor Cell Movement to the Lung in Setting of Pulmonary Hypertension

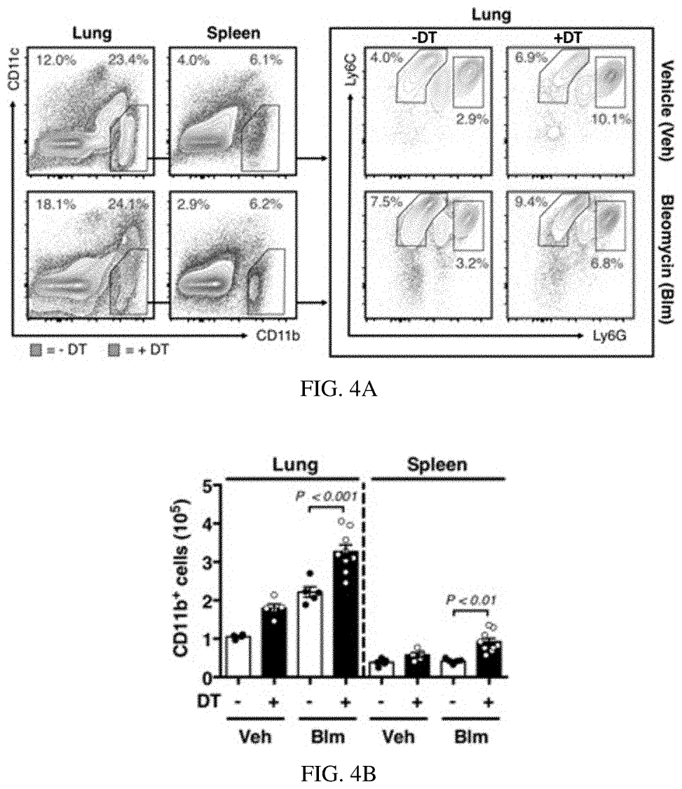

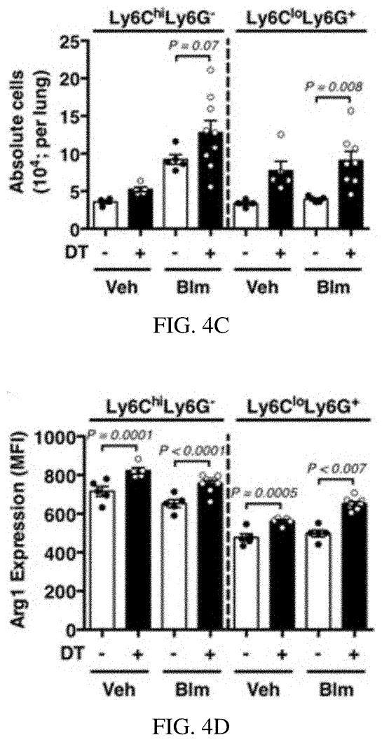

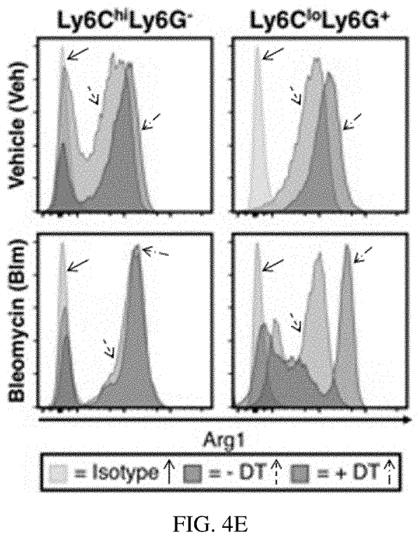

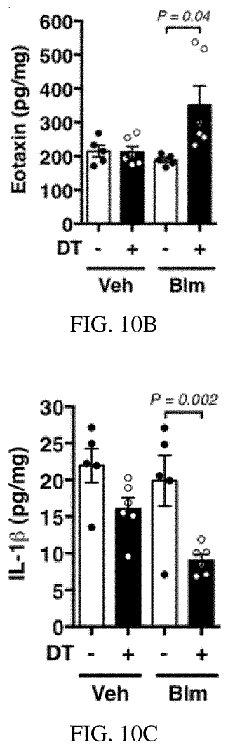

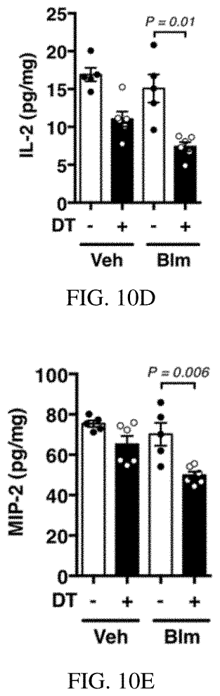

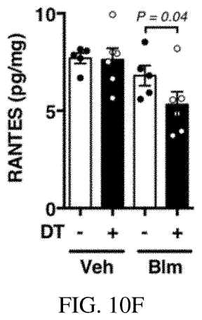

In order to assess and characterize evidence of emergency myelopoiesis in the model, lung and spleen from experimental mice were further examined for confirmation of increased CD11b⁺ cell subsets. Using flow cytometry, it was found that mice given DT and bleomycin displayed a large increase in CD11b⁺ absolute cell counts (FIGS. 4A and 4B), with a simultaneous rise in the number of pulmonary Ly6C^{hi}Ly6G⁻ (“monocytes”) and Ly6C^{lo}Ly6G⁺ (“neutrophils”) cells, although only significantly increased in the latter population (FIG. 4C). Consistent with these findings, when tissue homogenate was analyzed using a cytokine/chemokine array, expected granulocytic growth factors were elevated (G-CSF and Eotaxin), yet—surprisingly—other markers of inflammation were unexpectedly decreased in lungs from mice given DT, compared to bleomycin-treated controls (IL-1β, IL-2, MIP-2, and RANTES) (FIGS. 10A-10F).

It has previously been shown that such an immunosuppressive signature is associated with arginase 1 (Arg1) up-regulation particularly in neutrophil-like myeloid-derived cells, influenced largely by emergency myelopoiesis (Bryant et al., 2018). Therefore, levels of Arg1 were analyzed in the identified monocyte and neutrophil sub-populations, determining that protein expression was elevated in those cells from DT-treated mice, both vehicle and bleomycin-treated groups, compared to vehicle controls (FIGS. 4D and 4E).

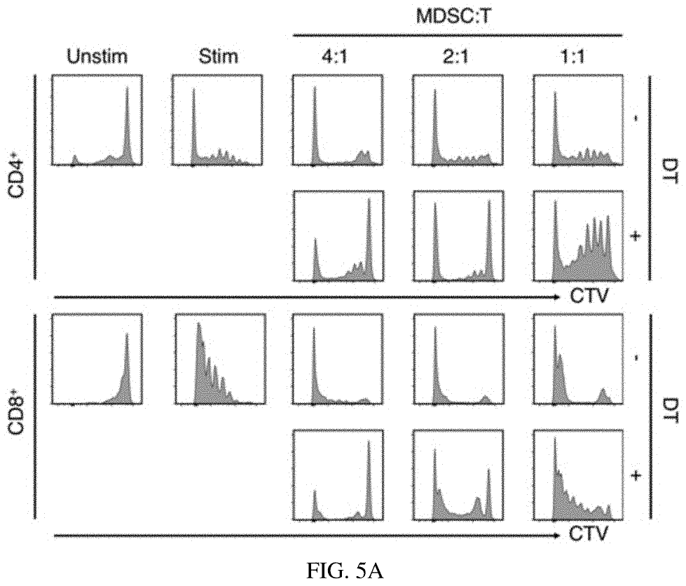

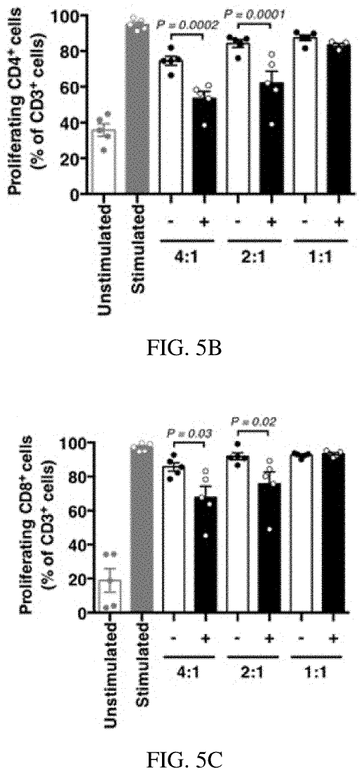

Closely related to neutrophils and monocytes in morphology, myeloid-derived suppressor cells (MDSC) are not normally present at high level in steady state, appearing in pathological conditions associated with chronic inflammation or stress. MDSC function, in part, by disrupting T cell function through generation of Arg1 in the microenvironment depleting availably arginine for normal metabolism (Gabrilovich, 2017). Therefore, it was of interest to determine if MDSC presence in the model could possibly account for the aforementioned immunosuppressive profile. Isolating Gr-1⁺ cells from either vehicle or DT-treated mDTR mice, “MDSC” were co-cultured with T cells in a dose-response fashion, examining for lymphocyte proliferation upon antibody-mediated stimulation (FIG. 5A). It was found that both CD4⁺ (FIG. 5B) and CD8⁺ (FIG. 5C) T cells were suppressed in response to DT-exposed myeloid-derived cells, compared to unstimulated controls. From these data, it was concluded that mDTR mice treated with DT and bleomycin undergo emergency myelopoiesis, ultimately yielding an increase in lung MDSC associated with PH.

### Example 2

### Immune Cell Expression of PD-1/PD-L1 is Enhanced in Pulmonary Hypertension Secondary to Pulmonary Fibrosis

With this understanding, it was sought to determine the contributory effects of myeloid cell PD-L1 expression on pulmonary vasculature in response to pulmonary fibrosis. To address this query, widespread, yet controlled, selective cellular apoptosis was induced in a model of pulmonary fibrosis with PH, reporting expression of PD-L1/PD-1 as a viable target to promote normal repair of the injured pulmonary circulation.

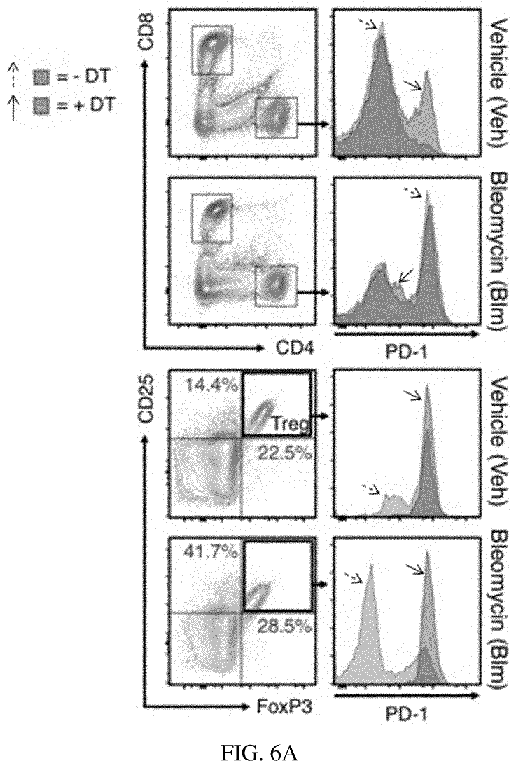

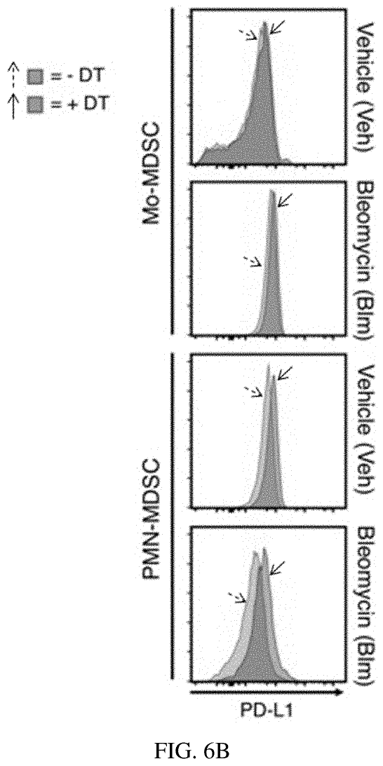

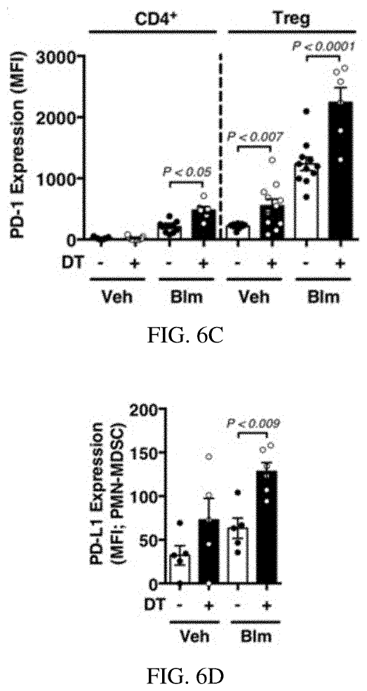

MDSC presence is known to be associated with T cell exhaustion and senescence via programmed cell death protein 1 (PD-1) upregulation on effector T cells, establishing the rationale for PD-1/programmed death-ligand 1 (PD-L1) signaling blockade as an effective immune therapy for many cancers (Huang, Francois, McGray, Miliotto & Odunsi, 2017). Thus, the level of PD-1 expression on T cell sub-populations was determined in this model of PH. Significant differences in CD4⁺ and CD4±CD25⁺FoxP3⁺ (Treg) cell expression of PD-1 were found (FIGS. 6A and 6C), the latter group displaying elevated PD-1 in both vehicle and bleomycin-exposed DT-treated mice lungs, compared to controls.

Vascular injury is known to lead to increased PD-L1 expression by circulating MDSC, with anti-PD-L1 therapy leading to subsequent improved T cell activation (Noman et al., 2014). In particular, MDSC facilitate an increase in exhaustive Treg in chronic inflammatory states, associated with increased PD-L1 expression (Lee et al., 2016). While there was no increase in PD-L1 expression by the monocytic MDSC (Mo-MDSC) subgroup, the neutrophilic MDSC (PMN-MDSC) population displayed a significant elevation in PD-L1 in the lungs of mice treated with both DT and bleomycin, compared to control animals (FIGS. 6B and 6D). These data support the conclusion that T cell PD-1—and PMN-MDSC PD-L1—expression is associated with development of worsening PH in the bleomycin-injury model.

### Anti-PD-L1 Prevents Development of Pulmonary Hypertension Secondary to Pulmonary Fibrosis Through Alteration in the Effector T Cell Response

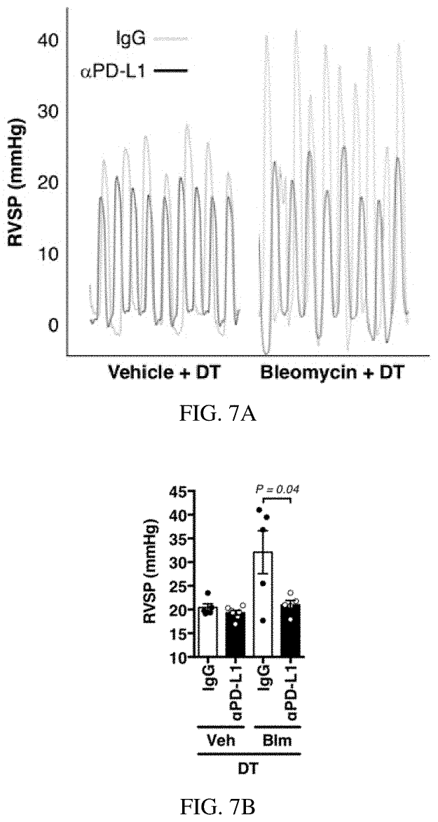

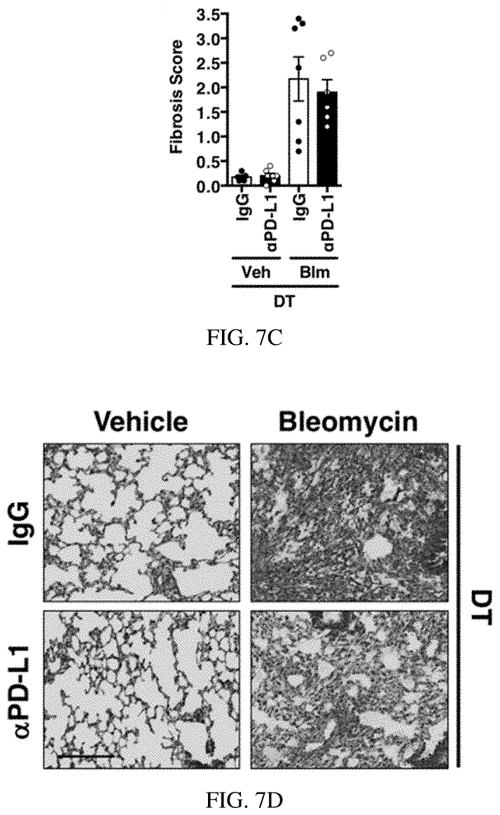

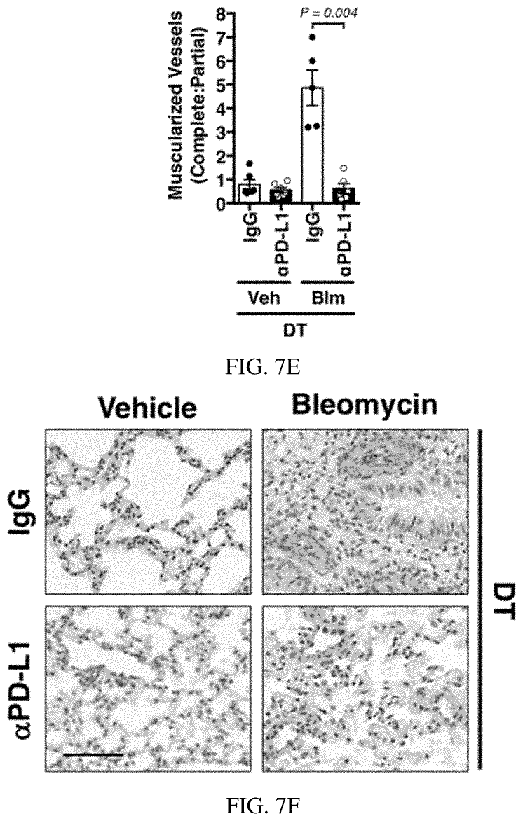

Next, in order to test PD-L1 as a viable therapeutic target for PH prevention, mDTR mice—all receiving DT—were treated with either anti-PD-L1 antibody (αPD-L1) or immunoglobulin control (IgG) using the bleomycin model. Mice given αPD-L1 preventively displayed near normal RVSP upon bleomycin treatment, compared to immunoglobulin treated controls (FIGS. 7A and 7B). Though there was no change in the degree of fibrosis (FIGS. 7C and 7D) between bleomycin treated groups, there was a corresponding decrease in ratio of fully to partially muscularized pulmonary vessels, compared to vehicle control-treated animal values (FIGS. 7E and 7F). These data highlight the absence of relationship between degree of fibrosis and development of pulmonary hypertension, a similar phenomenon to that seen in patients with IPF and PH.

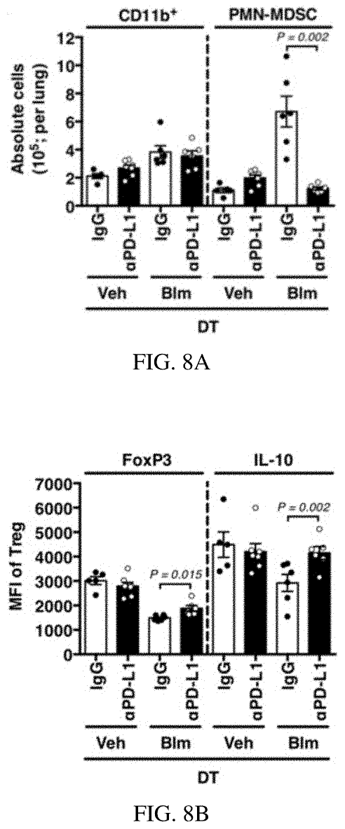

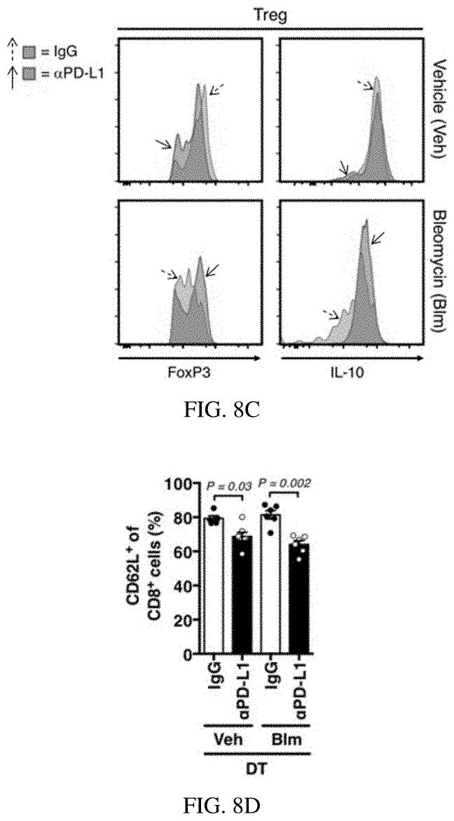

To explore a potential mechanism of αPD-L1 action, the effect of treatment on MDSC and T lymphocyte sub-groups was explored. First, it was shown that despite a lack of difference in absolute CD11b⁺ cell count between antibody-treated groups given bleomycin, there was a significant decrease in the number of lung PMN-MDSC with αPD-L1 injections (FIG. 8A). Interestingly, associated with this observed decrease was an increase in FoxP3 and IL-10 expression by pulmonary Treg in mice given αPD-L1 (FIGS. 8B and 8C). A decrease in CD62L⁺ cells was also noted—as a percentage of CD8⁺ effector T cells (FIG. 8D)—consistent with increased Treg capacity. Taken together, from these data it was concluded that the PD-1/PD-L1 signaling axis is a viable target for prevention of PH, potentially acting through alterations in a complex regulatory network of pulmonary PMN-MDSC and T lymphocytes.

### Example 3

Patients with Interstitial Lung Disease Complicated by Pulmonary Hypertension Display an Increase in PD-L1 Expression by Circulating Myeloid Cells

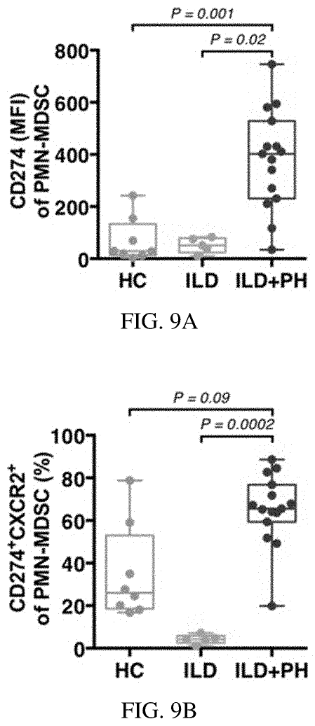

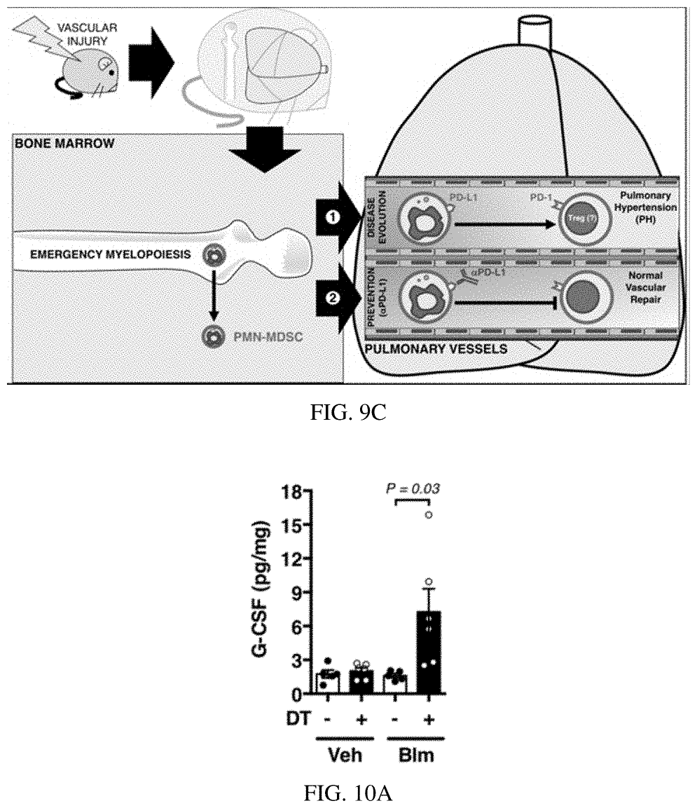

Given the preclinical relevance these studies suggest in conceivable treatment of patients with Group 3 PH, it was next sought to investigate differences in myeloid cell expression of PD-L1 (CD274) in peripheral blood samples from healthy controls (HC), and patients with interstitial lung disease with (ILD+PH) and without (ILD) PH. Using a previously described classification schema (Bronte et al., 2016), CD274 expression was quantified on CD33⁺CD11b⁺CD14⁻CD15⁺ cells (PMN-MDSC, human) and found that cells from patients with ILD+PH displayed higher levels of the checkpoint protein, compared to both controls and patients with simply ILD (FIG. 9A). These data are consistent with published work documenting an increase in CD274 expression by MDSC in a separate cohort of patients with pulmonary arterial hypertension (PAH) (Bryant et al., 2019).

Finally, chemokine receptor CXCR2 has been previously shown to act synergistically with PD-L1 in oncologic models of MDSC—specifically PMN-MDSC—recruitment and activation (Highfill et al., 2014). Moreover, CXCR2⁺ PMN-MDSC, acting directly through PD-1/PD-L1, induce CD4⁺ T cell exhaustion (Zhu, Gu, Xue, Yuan, Cao & Liu, 2017). Therefore, it was hypothesized that patients with ILD+PH would have more CD274⁺CXCR2⁺ PMN-MDSC, consistent with these prior observations. While the percentage of these dual-positive cells were not significantly increased compared to control subjects, there were markedly more CD274⁺CXCR2⁺ PMN-MDSC in the circulation of patients with ILD+PH, compared to those with just ILD (FIG. 9B). Cumulatively, these data indicate the applicability of the murine model (FIG. 9C) to the human pathologic condition, and represent a viable future therapeutic target for patients with disease.

### Discussion

As described herein, MDSC—particularly the polymorphonuclear subset (PMN-MDSC)—act to facilitate immune cell senescence in part through enhanced Arg1 production, primarily described in relation to either cancer (Romano et al., 2018) or autoimmune disease (Wu et al., 2016). The disease context of MDSC action is thus dependent upon specific T cell subgroup inhibition, illustrated by the changes in phenotype contingent upon Treg or Th17 cell proliferation/activation, that may act in concert with MDSC signaling (Hoechst, Gamrekelashvili, Manns, Greten & Korangy, 2011; Ji et al., 2016). For example, in irradiation-induced pneumonitis/fibrosis, Treg depletion alleviates lung inflammation invoking a see-saw effect on Th17 population, which is then elevated (Xiong et al., 2015); this has been observed in silica-induced pulmonary fibrosis, as well (Liu et al., 2010). The effect of the Treg:Th17 ratio is likely more complicated, however, with qualitative as well as quantitative measures contributing to disease progression, leading to seemingly contradictory findings depending on which cell population is targeted (Thakur et al., 2015). Illustrative of this concept is the finding that in patients with IPF, there is an increase in circulating Treg, and a decrease in Th17, similar to experimental results from cancer studies, but not those examining autoimmunity (Galati et al., 2014). A compensatory response to overcome an intrinsic defect in Treg function may be responsible, however, as another study demonstrated impaired suppressive capability by Treg from patients with IPF (Kotsianidis et al., 2009).

Similar findings have also been described in PH. For example, in rats exposed to hypoxia—inducing durable PH—those with an increase in Th17 compared to Treg cells developed more severe PH (Li et al., 2018). Conversely, in a clinical study of PAH patients, there was an increase in circulating Treg (an increase in the Treg:Th17 ratio) associated with worsened pulmonary vascular disease (Jasiewicz et al., 2016). Finally, in a patient population with PH related to connective tissue disease, a decrease in Treg population was associated with a worse prognosis and more severe PH (Gaowa et al., 2014). Moreover, the finding was worse in those with a documented depression in FEV1, potentially indicating patients with coexisting fibrotic lung disease. The results of the study discussed herein suggest that this delicate balance between T lymphocyte sub-populations is variably dependent upon PMN-MDSC associated PD-1/PD-L1 activity (Limagne et al., 2016).

Given prior reports on abrogation of the PH response in a similar model of disease (Bryant et al., 2018), by merely blocking accumulation of MDSC, it is not believed that such a loss of native cell populations is playing a large part in the observed maladaptive pulmonary vascular changes.

In conclusion, these results indicate the importance of PD-L1 targeting in PH patients. The anti-PDL1 therapies provided herein represent feasible and safe treatments for a debilitating disease that currently has none.

### Experimental Methods

### Animals

LysM.Cre (stock 004781), iDTR (stock 007900), and C57BL/6J (stock 000664) were purchased from the Jackson Laboratory. LysM.Cre and homozygous iDTR were crossed to generate LysM.Cre-DTR (“mDTR”) mice, respectively, for experiments as previously described (Goren et al., 2009) All mice were greater than 10 weeks of age at the study onset, included both males and females, and ranged in weight from 20 to 30 g. All animal studies were approved by the University of Florida Institutional Animal Care and Use Committee (IACUC; Protocol 08702). Studies involving animals are reported in accordance with the ARRIVE guidelines for reporting experiments involving animals (Kilkenny, Browne, Cuthill, Emerson & Altman, 2010).

### Statistical Analysis

All graphing and statistical analyses were carried out using GraphPad Prism (GraphPad Software). All animal data are presented as mean±SEM. The Student's t test was used for single comparisons, and 2-way ANOVA was used for multiple comparisons. Human data are presented as median±IQR. The Mann-Whitney U test or the Kruskal-Wallis rank-sum test was used for non-normal data, with Dunn's multiple comparison test. P<0.05 was considered significant.

### Bleomycin-Induced Pulmonary Fibrosis and Pulmonary Hypertension

Mice underwent intraperitoneal injection with 0.018 U/g bleomycin (Thermo Fisher Scientific) or vehicle (PBS) twice weekly for 4 weeks (Pi et al., 2018). For diphtheria toxin (DT) studies, mice were injected intraperitoneally for three consecutive days with 100 ng of diphtheria toxin (DT; SigmaAldrich) before initiation of bleomycin protocol, with PBS injection used as control (Goren et al., 2009), and twice weekly thereafter. Additionally, anti-PD-L1 (500 □g, intraperitoneally) or rat IgG2b, both purchased from BioXcell, were administered on Day 0, then once weekly for three additional doses (Celada et al., 2018) throughout bleomycin exposure.

### Flow Cytometry, Antibodies, and Multiplex Array

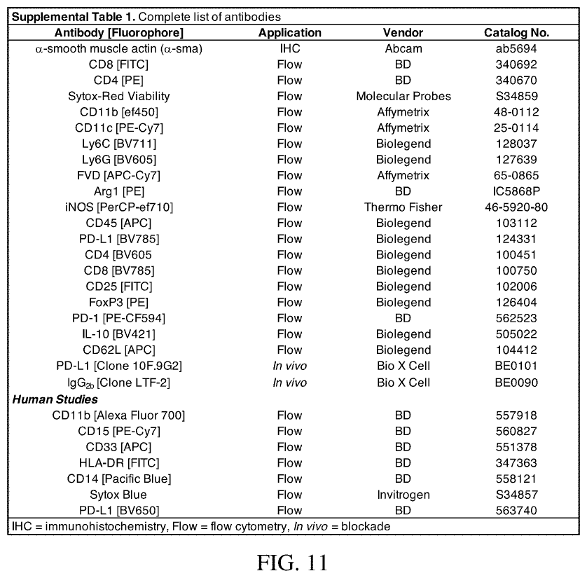

Flow cytometry analyses were performed on a BD LSR II or on FACSCalibur upgraded at three lasers and 8 colors (Cytek). Cell populations were identified using sequential gating strategy characterized within body of manuscript (excluding debris and doublets). Fluorescence minus one (FMO) and isotype controls were used when necessary. The expression of markers is presented as median fluorescence intensity (MFI). Data were analyzed using FlowJo software (Tree Star). A comprehensive list of antibodies used in the experiments is provided in FIG. 11, and as detailed previously (Bryant et al., 2018; Misharin, Morales-Nebreda, Mutlu, Budinger & Perlman, 2013). A multiplex array (Millipore) was used to detect and quantify mouse cytokine/chemokines in whole lung homogenate using previously described techniques (Bryant et al., 2018). Data were acquired using a Luminex®200™ and analyzed using Milliplex Analyst Software (VigeneTech Inc).

### Pulmonary Hemodynamic and Histologic Assessments

Upon completion of experimental protocols, intact mice underwent invasive closed-chest measurement of right ventricular systolic pressure (RVSP). In brief, a Millar 1.4 French pressure-volume microtip catheter transducer (SPR-839; Millar Instruments) connected to a PowerLab/8s (ADInstruments) was inserted through a right internal jugular vein incision and threaded down into the right ventricle. RVSP (mmHg) recordings were collected using Chart 5 (ADInstruments). Upon completion of the measurements, the heart was excised with removal of the atria, and the RV and left ventricle (LV) plus septum, isolated for measurement of the RV:LV+S as previously described (Hemnes et al., 2014). Formalin-fixed, paraffin-embedded, was then assessed histologic fibrosis score (Tanjore et al., 2013). Lung histology was additionally stained for α-smooth muscle actin and assessed for muscularized pulmonary vessel count (Bryant et al., 2015). Images were obtained using a Keyence BZ-X microscope, with analysis performed using BZ-X Analyzer software (Keyence).

### T-Cell Proliferation Assay

T-cell proliferation/suppression assays were performed as previously described (Highfill et al., 2014). In brief, wild-type C57BL/6 T cells were isolated with CD3 monoclonal antibody-coated magnetic beads (Miltenyi Biotec), and stained with CTV. These cells were then stimulated with anti-CD3/CD28 mouse beads (ThermoFisher). The ratio between splenic MDSC and T-cells was tested at 4:1, 2:1, and 1:1. Cells were incubated for five days, with proliferation assessed as CTV dilution by flow cytometry, and percent proliferation calculated for each group.

### Study Approval

Human sample use was approved by the Institutional Review Board (IRB) of Rhode Island Hospital. Subjects were enrolled from the Rhode Island Hospital Pulmonary Hypertension Center as part of a local registry and biorepository (Baird et al., 2018), which captures all patients referred for PH evaluation. Subjects were included if they were clinically phenotyped as pulmonary fibrosis with no PH or pulmonary fibrosis with PH by standard hemodynamic and/or echocardiographic criteria, as previously described (Ventetuolo et al., 2016).

### Example 4—Clinical Trial

### Objectives

A primary objective of the MIC Check clinical trial was to assess safety of PD-L1 inhibitor therapy among patients with ILD and PH. A secondary objective was to compare potential surrogate biomarkers of clinical disease progression in the two groups, including MDSC sub-population expansion (e.g. PMN-MDSC sub-population expansion) and T-cell characterization. Another secondary objective was to assess the effect of therapy on cardiopulmonary function in each group. A tertiary objective was to assess mortality in each group.

### Hypotheses

The primary hypothesis is that PD-1/PD-L1 signaling, through MDSC, contributes to progression of PH in patients with ILD.

### Design

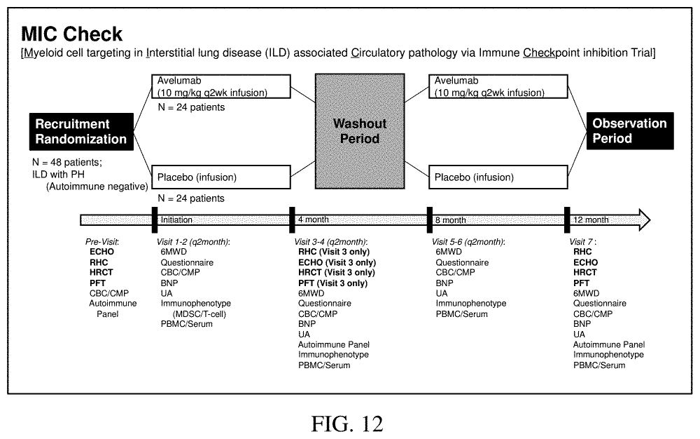

Double-blind, randomized, crossover, placebo-controlled clinical trial (see FIG. 12). The trial will be conducted in three separate studies, where patients in the first study receive a 1,200 mg infusion of atezolizumab (Tecentriq®), or placebo, every 3 weeks; patients in the second study receive a 200 mg infusion of pembrolizumab (Keytruda®), or placebo, every 3 weeks; and patients in the third study receive a 10 mg/kg infusion of avelumab (Bavencio®), or placebo, every 2 weeks. Each trial will have a washout period before the crossover.

### Patient Population

Interstitial lung disease with pulmonary hypertension (Group III PH). The size of the trial is 48 patients in each study, with 24 receiving antibody and 24 receiving placebo.

### Methods

### Inclusion Criteria

Consensus diagnosis of interstitial lung disease with right heart catheter confirmed pulmonary hypertension. Patients with autoimmune diseases will be ineligible.

### Exclusion Criteria

Pulmonary artery occlusion pressure >15 mmHg consistent with Group II PH. Clinical or laboratory diagnosis of autoimmune-related illness, including CT-ILD (connective tissue disease associated with ILD) and IPAF (interstitial pneumonia with autoimmune features).

### Outcomes Measures/Analysis

Primary outcome is assessment of safety and tolerability of PD-1/PD-L1 therapy in patients with ILD and PH. Secondary outcomes include assessment of parenchymal lung disease by pulmonary function tests (PFTs) and High Resolution Computed Tomography (HRCT) and pulmonary vascular response by right heart catheterization (RHC) to immune checkpoint inhibitor therapy (see FIG. 12). In particular, patient analysis and monitoring will include, but will not be limited to, the following:
Pre-visit: Patients' vascular health will be assessed by echocardiogram (ECHO), RHC, HRCT, PFTs, autoimmune panel and comprehensive metabolic panel (CMP) and complete blood count (CBC) tests.
Initiation period, or 0-1 months after trial begins: Patients will be assessed by 6-minute walk distance test (6MWD), a questionnaire, CBC/CMP, Brain natriuretic peptide (BNP) test, urinalysis (UA), peripheral blood mononuclear cells (PBMC) and serum analysis, and an immunophenotype screen based on absolute counts of circulating MDSC cells and PD-L1 expression levels on MDSC.
4 months after trial begins: Patients will be assessed by RHC, ECHO, HRCT, PFT, 6MWD, questionnaires, CBC/CMP, BNP, UA, PMBC/serum analyses, autoimmune panel and immunophenotype screens.
8 months after trial begins, and after Washout Period: Patients will be assessed by 6MWD, questionnaire, CBC/CMP, BNP, UA, immunophenotype screen and PMBC/serum analyses.
12 months after trial begins: Patients will be assessed by RHC, ECHO, HRCT, PFT, 6MWD, questionnaires, CBC/CMP, BNP, UA, PMBC/serum analyses, autoimmune panel and immunophenotype screens.

### REFERENCES

Al Sayed M F, Amrein M A, Buhrer E D, Huguenin A L, Radpour R, Riether C, et al. (2019). T-cell-Secreted TNFalpha Induces Emergency Myelopoiesis and Myeloid-Derived Suppressor Cell Differentiation in Cancer. Cancer Res 79: 346-359.
Asosingh, K., et al. Pulmonary vascular disease in mice xenografted with human B M progenitors from patients with pulmonary arterial hypertension. Blood 120, 1218-1227 (2012).
Baird G L, Archer-Chicko C, Barr R G, Bluemke D A, Foderaro A E, Fritz J S, et al. (2018). Lower DHEA-S levels predict disease and worse outcomes in post-menopausal women with idiopathic, connective tissue disease- and congenital heart disease-associated pulmonary arterial hypertension. Eur Respir J 51.
Ballbach M, Dannert A, Singh A, Siegmund D M, Handgretinger R, Piali L, et al. (2017). Expression of checkpoint molecules on myeloid-derived suppressor cells. Immunol Lett 192: 1-6.
Boettcher S, Gerosa R C, Radpour R, Bauer J, Ampenberger F, Heikenwalder M, et al. (2014). Endothelial cells translate pathogen signals into G-CSF-driven emergency granulopoiesis. Blood 124: 1393-1403.
Bronte V, Brandau S, Chen S H, Colombo M P, Frey A B, Greten T F, et al. (2016). Recommendations for myeloid-derived suppressor cell nomenclature and characterization standards. Nat Commun 7: 12150.
Bryant A J, Fu C, Lu Y, Brantly M L, Mehrad B, Moldawer L L, et al. (2019). A checkpoint on innate myeloid cells in pulmonary arterial hypertension. Pulm Circ 9(1): 1-5.
Bryant A J, Shenoy V, Fu C, Marek G, Lorentsen K J, Herzog E L, et al. (2018). Myeloid-derived Suppressor Cells Are Necessary for Development of Pulmonary Hypertension. Am J Respir Cell Mol Biol 58: 170-180.
Bryant A J, Robinson L J, Moore C S, Blackwell T R, Gladson S, Penner N L, et al. (2015). Expression of mutant bone morphogenetic protein receptor II worsens pulmonary hypertension secondary to pulmonary fibrosis. Pulm Circ 5: 681-690.
Burton V J, Holmes A M, Ciuclan L I, et al. Attenuation of leukocyte recruitment via CXCR1/2 inhibition stops the progression of PAH in mice with genetic ablation of endothelialBMPR-II. Blood 2011; 118: 4750-4758.
Celada L J, Kropski J A, Herazo-Maya J D, Luo W, Creecy A, Abad A T, et al. (2018). PD-1 up-regulation on CD4(+) T cells promotes pulmonary fibrosis through STAT3-mediated IL-17A and TGF-beta1 production. Sci Transl Med 10.
Chiba Y, Mizoguchi I, Hasegawa H, Ohashi M, Orii N, Nagai T, et al. (2018). Regulation of myelopoiesis by proinflammatory cytokines in infectious diseases. Cell Mol Life Sci 75: 1363-1376.
Farha S., et al. Hypoxia-inducible factors in human pulmonary arterial hypertension: a link to the intrinsic myeloid abnormalities. Blood 117, 3485-3493 (2011).
Gabrilovich D I (2017). Myeloid-Derived Suppressor Cells. Cancer Immunol Res 5: 3-8.
Galati D, De Martino M, Trotta A, Rea G, Bruzzese D, Cicchitto G, et al. (2014). Peripheral depletion of NK cells and imbalance of the Treg/Th17 axis in idiopathic pulmonary fibrosis patients. Cytokine 66: 119-126.
Gaowa S, Zhou W, Yu L, Zhou X, Liao K, Yang K, et al. (2014). Effect of Th17 and Treg axis disorder on outcomes of pulmonary arterial hypertension in connective tissue diseases. Mediators Inflamm 2014: 247372.
Geng Y, Liu X, Liang J, Habiel D M, Kulur V, Coelho A L, et al. (2019). PD-L1 on invasive fibroblasts drives fibrosis in a humanized model of idiopathic pulmonary fibrosis. JCI Insight 4.
Goren I, Allmann N, Yogev N, Schurmann C, Linke A, Holdener M, et al. (2009). A transgenic mouse model of inducible macrophage depletion: effects of diphtheria toxin-driven lysozyme M-specific cell lineage ablation on wound inflammatory, angiogenic, and contractive processes. Am J Pathol 175: 132-147.
Hemnes A R, Brittain E L, Trammell A W, Fessel J P, Austin E D, Penner N, et al. (2014). Evidence for right ventricular lipotoxicity in heritable pulmonary arterial hypertension. Am J Respir Crit Care Med 189: 325-334.
Highfill S L, Cui Y, Giles A J, Smith J P, Zhang H, Morse E, et al. (2014). Disruption of CXCR2-mediated MDSC tumor trafficking enhances anti-PD1 efficacy. Sci Transl Med 6: 237ra267.
Hoechst B, Gamrekelashvili J, Manns M P, Greten T F, & Korangy F (2011). Plasticity of human Th17 cells and iTregs is orchestrated by different subsets of myeloid cells. Blood 117: 6532-6541.
Huang R Y, Francois A, McGray A R, Miliotto A, & Odunsi K (2017). Compensatory upregulation of PD-1, LAG-3, and CTLA-4 limits the efficacy of single-agent checkpoint blockade in metastatic ovarian cancer. Oncoimmunology 6: e1249561.
Huertas A., et al. Regulatory T Cell Dysfunction in Idiopathic, Heritable and Connective Tissue-Associated Pulmonary Arterial Hypertension. Chest 149, 1482-1493 (2016).
Jasiewicz M, Moniuszko M, Pawlak D, Knapp M, Rusak M, Kazimierczyk R, et al. (2016). Activity of the kynurenine pathway and its interplay with immunity in patients with pulmonary arterial hypertension. Heart 102: 230-237.
Ji J, Xu J, Zhao S, Liu F, Qi J, Song Y, et al. (2016). Myeloid-derived suppressor cells contribute to systemic lupus erythaematosus by regulating differentiation of Th17 cells and Tregs. Clin Sci (Lond) 130: 1453-1467.
Johnson D B, Balko J M, Compton M L, Chalkias S, Gorham J, Xu Y, et al. (2016). Fulminant Myocarditis with Combination Immune Checkpoint Blockade. N Engl J Med 375: 1749-1755.
Kilkenny C, Browne W J, Cuthill I C, Emerson M, & Altman D G (2010). Improving bioscience research reporting: the ARRIVE guidelines for reporting animal research. PLoS Biol 8: e1000412.
Kim S H, et al., Phenformin Inhibits Myeloid-Derived Suppressor Cells and Enhances the Anti-Tumor Activity of PD-1 Blockade in Melanoma. J. Invest. Dermatol. (2017) 137:1740-1748.
Kinder B W, Brown K K, Schwarz M I, Ix J H, Kervitsky A, & King T E, Jr. (2008). Baseline BAL neutrophilia predicts early mortality in idiopathic pulmonary fibrosis. Chest 133: 226-232.
Kotsianidis I, Nakou E, Bouchliou I, Tzouvelekis A, Spanoudakis E, Steiropoulos P, et al. (2009). Global impairment of CD4+CD25+FOXP3+ regulatory T cells in idiopathic pulmonary fibrosis. Am J Respir Crit Care Med 179: 1121-1130.
Lee C R, Kwak Y, Yang T, Han J H, Park S H, Ye M B, et al. (2016). Myeloid-Derived Suppressor Cells Are Controlled by Regulatory T Cells via TGF-beta during Murine Colitis. Cell Rep 17: 3219-3232.
Li C, Liu P P, Tang D D, Song R, Zhang Y Q, Lei S, et al. (2018). Targeting the RhoA-ROCK pathway to regulate T-cell homeostasis in hypoxia-induced pulmonary arterial hypertension. Pulm Pharmacol Ther 50: 111-122.
Limagne E, Euvrard R, Thibaudin M, Rebe C, Derangere V, Chevriaux A, et al. (2016). Accumulation of MDSC and Th17 Cells in Patients with Metastatic Colorectal Cancer Predicts the Efficacy of a FOLFOX-Bevacizumab Drug Treatment Regimen. Cancer Res 76: 5241-5252.
Liu F, Liu J, Weng D, Chen Y, Song L, He Q, et al. (2010). CD4+CD25+Foxp3+ regulatory T cells depletion may attenuate the development of silica-induced lung fibrosis in mice. PLoS One 5: e15404.
Loftus T J, Mohr A M, & Moldawer L L (2018). Dysregulated myelopoiesis and hematopoietic function following acute physiologic insult. Curr Opin Hematol 25: 37-43.
McGoon, M. D., et al. Pulmonary arterial hypertension: epidemiology and registries. J Am Coll Cardiol 62, D51-59 (2013).
Misharin A V, Morales-Nebreda L, Mutlu G M, Budinger G R, & Perlman H (2013). Flow cytometric analysis of macrophages and dendritic cell subsets in the mouse lung. Am J Respir Cell Mol Biol 49: 503-510.
Noman M Z, Desantis G, Janji B, Hasmim M, Karray S, Dessen P, et al. (2014). PD-L1 is a novel direct target of HIF-lalpha, and its blockade under hypoxia enhanced MDSC-mediated T cell activation. J Exp Med 211: 781-790.
Pi L, Fu C, Lu Y, Zhou J, Jorgensen M, Shenoy V, et al. (2018). Vascular Endothelial Cell-Specific Connective Tissue Growth Factor (CTGF) Is Necessary for Development of Chronic Hypoxia-Induced Pulmonary Hypertension. Front Physiol 9: 138.
Romano A, Parrinello N L, La Cava P, Tibullo D, Giallongo C, Camiolo G, et al. (2018). PMN-MDSC and arginase are increased in myeloma and may contribute to resistance to therapy. Expert Rev Mol Diagn 18: 675-683.
Shimomura I, Abe M, Li Y, Tsushima K, Sakao S, Tanabe N, et al. (2019). Pulmonary Hypertension Exacerbated by Nintedanib Administration for Idiopathic Pulmonary Fibrosis. Intern Med 58: 965-968.
Strauss R G (2015). Neutrophil/granulocyte transfusions collected from G-CSF+ dexamethasone-stimulated donors. Curr Opin Hematol 22: 565-567.
Taichman D B & Mandel, J Epidemiology of pulmonary arterial hypertension. Clin Chest Med 34, 619-637 (2013).
Tanjore H, Degryse A L, Crossno P F, Xu X C, McConaha M E, Jones B R, et al. (2013). beta-catenin in the alveolar epithelium protects from lung fibrosis after intratracheal bleomycin. American journal of respiratory and critical care medicine 187: 630-639.
Taylor S, Dirir O, Zamanian R T, Rabinovitch M, & Thompson A A R (2018). The Role of Neutrophils and Neutrophil Elastase in Pulmonary Arterial Hypertension. Front Med (Lausanne) 5: 217.
Thakur C, Wolfarth M, Sun J, Zhang Y, Lu Y, Battelli L, et al. (2015). Oncoprotein mdig contributes to silica-induced pulmonary fibrosis by altering balance between Th17 and Treg T cells. Oncotarget 6: 3722-3736.
van Amerongen M J, Harmsen M C, van Rooijen N, Petersen A H, & van Luyn M J (2007). Macrophage depletion impairs wound healing and increases left ventricular remodeling after myocardial injury in mice. Am J Pathol 170: 818-829.
Ventetuolo C E, Baird G L, Barr R G, Bluemke D A, Fritz J S, Hill N S, et al. (2016). Higher Estradiol and Lower Dehydroepiandrosterone-Sulfate Levels Are Associated with Pulmonary Arterial Hypertension in Men. Am J Respir Crit Care Med 193: 1168-1175.
Watanabe R, Shirai T, Namkoong H, Zhang H, Berry G J, Wallis B B, et al. (2017). Pyruvate controls the checkpoint inhibitor PD-L1 and suppresses T cell immunity. J Clin Invest 127: 2725-2738.
Wu H, Zhen Y, Ma Z, Li H, Yu J, Xu Z G, et al. (2016). Arginase-1-dependent promotion of TH17 differentiation and disease progression by MDSCs in systemic lupus erythematosus. Sci Transl Med 8: 331ra340.
Xiong S, Guo R, Yang Z, Xu L, Du L, Li R, et al. (2015). Treg depletion attenuates irradiation-induced pulmonary fibrosis by reducing fibrocyte accumulation, inducing Th17 response, and shifting IFN-gamma, IL-12/IL-4, IL-5 balance. Immunobiology 220: 1284-1291.
Yan L, et al. Bone Marrow-derived Cells Contribute to the Pathogenesis of Pulmonary Arterial Hypertension. Am J Respir Crit Care Med 193, 898-909 (2016).
Yeager M E, Nguyen C M, Belchenko D D, Colvin K L, Takatsuki S, Ivy D D, et al. (2012). Circulating myeloid-derived suppressor cells are increased and activated in pulmonary hypertension. Chest 141: 944-952.
Zhu H, Gu Y, Xue Y, Yuan M, Cao X, & Liu Q (2017). CXCR2(+) MDSCs promote breast cancer progression by inducing EMT and activated T cell exhaustion. Oncotarget 8: 114554-114567.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value. Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating pulmonary hypertension, the method comprising administering a PD-L1 inhibitor or PD-1 inhibitor to a subject having pulmonary hypertension.

2. The method of claim 1, wherein the PD-L1 inhibitor or PD-1 inhibitor is a monoclonal antibody.

3. The method of claim 2, wherein the antibody is pembrolizumab, atezolizumab, avelumab, nivolumab, or durvalumab.

4-8. (canceled)

9. The method of claim 1, wherein the step of administering comprises contacting myeloid-derived suppressor cells (MDSC) of the lung of the subject with the PD-L1 inhibitor or PD-1 inhibitor.

10. The method of claim 1, wherein the subject has interstitial lung disease.

11. The method of claim 2, wherein the antibody is administered semi-weekly.

12. The method of claim 2, wherein the antibody is administered every three weeks.

13. The method of claim 2, wherein the antibody is administered in a dose of about 10 mg/kg of subject.

14. The method of claim 2, wherein the antibody is administered in a dose of about 200 mg to about 1200 mg.

15. The method of claim 1 further comprising administering a CXCR2 inhibitor.

16. (canceled)

17. The method of claim 1, wherein the subject has previously been treated for pulmonary hypertension.

18. The method of claim 1, wherein the PD-L1 inhibitor or PD-1 inhibitor is administered in a pharmaceutical composition comprising one or more additional pharmaceutically acceptable agents.

19. The method of claim 1, wherein the subject is human.

20. (canceled)

21. A method for identifying a subject having pulmonary hypertension that is sensitive to treatment with a PD-L1 inhibitor or a PD-1 inhibitor, the method comprising:
(a) counting the absolute number of polymorphonuclear MDSC (PMN-MDSC) in a first biological sample obtained from a subject having a pulmonary hypertension;
(b) administering a PD-L1 inhibitor or a PD-1 inhibitor to the subject;
(c) counting the absolute number of PMN-MDSC in a second biological sample obtained from the subject; and
(d) identifying the subject as having pulmonary hypertension that is sensitive to treatment with a PD-L1 inhibitor or a PD-1 inhibitor if the absolute number generated in (c) is not substantially lower than the absolute number generated in (a).

22-28. (canceled)

29. A method for identifying a subject having a pulmonary hypertension that is sensitive to treatment with a PD-L1 inhibitor or a PD-1 inhibitor, the method comprising:
(a) detecting the expression levels of PD-1 receptor on the surface of PMN-MDSC in a biological sample obtained from a subject having a pulmonary hypertension; and
(b) identifying the subject as having a pulmonary hypertension that is sensitive to treatment with a PD-L1 inhibitor or a PD-1 inhibitor if the expression levels are substantially lower than normal controls.

30. (canceled)

31. A method for identifying a subject having a pulmonary hypertension that is sensitive to treatment with a CXCR2 inhibitor, the method comprising:
(a) detecting the expression levels of CXCR2 receptor on the surface of PMN-MDSC in a biological sample obtained from a subject having a pulmonary hypertension; and
(b) identifying the subject as having a pulmonary hypertension that is sensitive to treatment with a CXCR2 inhibitor if the expression levels are substantially lower than normal controls.

32. (canceled)

33. A method for identifying a subject having a pulmonary hypertension that is sensitive to treatment with a PD-L1 inhibitor or a PD-1 inhibitor, the method comprising:
(a) detecting the counts of circulating PMN-MDSC in a biological sample obtained from a subject having a pulmonary hypertension; and
(b) identifying the subject as having a pulmonary hypertension that is sensitive to treatment with a PD-L1 inhibitor or a PD-1 inhibitor if the counts are substantially higher than normal controls.

34-38. (canceled)

39. The method of claim 1, wherein the method does not comprise a step of administering a phosphodiesterase-5 inhibitor.

40. The method of claim 1, wherein the PD-L1 inhibitor or PD-1 inhibitor does not comprise a monoclonal antibody.

41. The method of claim 2, wherein the antibody does not comprise nivolumab, pembrolizumab, or atezolizumab.
